(19) United States
(12) Patent Application Publication
(10) Pub. No.: US 2018/0344836 A1
(43) Pub. Date: Dec. 6, 2018

(54) METHODS FOR TREATING, PREVENTING AND DIAGNOSING PORCINE EPIDEMIC DIARRHEA VIRUS INFECTION

(71) Applicant: University of Saskatchewan, Saskatoon (CA)

(72) Inventors: Volker GERDTS, Saskatoon (CA); Brenda ALLAN, Saskatoon (CA); Jan VAN DEN HURK, Saskatoon (CA); Yan ZHOU, Saskatoon (CA); Alexander ZAKHARTCHOUK, Saskatoon (CA); Suresh TIKOO, Saskatoon (CA); Andrew POTTER, Saskatoon (CA)

(73) Assignee: University of Saskatchewan, Saskatoon, SK (CA)

(21) Appl. No.: 15/779,509

(22) PCT Filed: Nov. 24, 2016
(86) PCT No.: PCT/CA2016/051381

§ 371 Date: May 25, 2018

Related U.S. Application Data
(60) Provisional application No. 62/382,395, filed on Sep. 1, 2016.
(60) Provisional application No. 62/260,128, filed on Nov. 25, 2015.

(51) Int. Cl. A61K 39/12; A61K 39/39; G01N 33/569; A61P 31/12

(52) U.S. Cl. CPC A61K 39/12; A61K 39/39; A61K 2039/5252; A61P 31/12; G01N 33/56983

(57) ABSTRACT

Immunogens, compositions and methods for treating, preventing and/or diagnosing PEDV infection in pigs are disclosed. The compositions and methods use inactivated or attenuated virus-containing vaccines, or subunit vaccines, including immunogens and mixtures of immunogens derived from PEDV isolates.

### TECHNICAL FIELD

The present invention relates generally to viral pathogens. In particular, the invention pertains to Porcine Epidemic Diarrhea Virus (PEDV) and methods of treating, preventing and/or diagnosing PEDV infection and PEDV-related disorders in pigs.

### BACKGROUND

Porcine Epidemic Diarrhea Virus (PEDV) is a member of the Coronaviridae family and is an enveloped, single-stranded, positive-sense RNA virus. PEDV has an approximately 28 kb genome that encodes non-structural proteins and four major structural proteins including spike, envelope, membrane, and nucleocapsid proteins (Song, et al., Virus Genes (2012) 44:167-175). PEDV causes severe diarrhea and dehydration in pigs. The virus is often fatal to newborn piglets. Adult pigs typically become sick and experience weight loss and sometimes death when infected.

PEDV was first discovered in the early 1970's in the United Kingdom. The virus has since spread to many parts of Europe, Asia and North America and over the last 10 years has caused severe economic losses in Asia including China, Japan, Thailand and South Korea. The virus was first discovered in a North American herd in April of 2013. Since then, PEDV has been identified on more than 8,000 farms in more than 30 States in the United States and various parts of Mexico and has caused outbreaks of severe diarrhea in young piglets with high mortality. The first outbreak in Canada appeared in 2014, and since then more than 100 outbreaks have been described. All of these outbreaks have been contained through biosecurity and management. However, the threat of this disease becoming endemic in North American herds is enormous and large resources have been put into efforts to control the disease including early and rapid detection of the virus, disease surveillance and enhanced biosecurity.

The mode of PEDV transmission is typically fecal-oral; however it also appears the virus has the ability to aerosolize and be transported over large distances by air.

It is clear that PEDV is rapidly becoming a major threat to the health of swine worldwide. Due to the tremendous economic impact of PEDV, compositions and methods of treating, preventing and/or diagnosing infection are needed.

### SUMMARY OF THE INVENTION

The inventors herein have developed an inactivated vaccine for PEDV that has proven safe and highly effective in newborn piglets. When administered to sows four and two weeks prior to farrowing, vaccination resulted in high levels of antigen-specific colostral and milk SIgA- and IgG-antibodies in piglets born to vaccinated sows. High levels of virus neutralizing antibodies were found in serum of piglets born to vaccinated sows. Surprisingly, as high as 95% of all vaccinated piglets survived infection and showed significantly reduced clinical symptoms, reduced weight loss and reduced viral shedding. In contrast, all control animals displayed severe clinical symptoms, including severe weight loss and dehydration, and approximately 50% of these piglets died within 6 days post infection. These results show that the vaccine described herein is highly effective against PEDV disease and in particular, the neonatal form of the disease.

Additionally, compositions comprising isolated immunogens from the PEDV polyprotein non-structural regions are described, as are particular epitopes from the PEDV nucleocapsid protein. These immunogens are useful for preventing, treating and diagnosing PEDV infection.

Thus, the present invention relates to the use of PEDV preparations in the treatment and/or prevention of PEDV infection in pigs. Attenuated or inactivated virus-containing vaccines, or subunit vaccines, including immunogens and mixtures of immunogens derived from PEDV isolates, are used to provide protection against subsequent infection with PEDV and/or to diagnose PEDV infection. The present invention thus provides a commercially useful method of treating, preventing and/or diagnosing PEDV infection in swine.

Accordingly, in one embodiment, a composition is provided that comprises an inactivated or attenuated Porcine Epidemic Diarrhea Virus (PEDV) or one or more isolated PEDV immunogens; a pharmaceutically acceptable vehicle; and an immunological adjuvant. In certain embodiments, the immunological adjuvant is selected from (a) alum or (b) an adjuvant composition comprising a host defense peptide, an immunostimulatory sequence, such as a CpG or poly (I:C), and a polyphosphazine. In certain embodiments, the polyphosphazine is selected from poly[di(sodium carboxylatophenoxy)phosphazene] (PCPP), poly(di-4-oxyphenylproprionate)phosphazene (PCEP), or a PCPP polymer comprising 90% PCPP copolymer with 10% hydroxyl groups (90:10 PCPP).

In further embodiments, the PEDV has a genomic cDNA sequence with at least 90% sequence identity to SEQ ID NO:1.

In additional embodiments, the attenuated PEDV in the compositions comprises a mutation in a sequence of amino acids corresponding to SEQ ID NOS:28, 29 and/or 30.

In yet further embodiments, a composition is provided that comprises (a) at least one isolated immunogen comprising an epitope from a PEDV spike (5) protein, a PEDV ORF3 protein, a PEDV envelope (E) protein, a PEDV membrane (M) protein, and/or a PEDV nucleocapsid (N) protein; (b) a pharmaceutically acceptable vehicle; and (c) an immunological adjuvant.

In certain embodiments, the isolated immunogen is an isolated PEDV nucleocapsid immunogen, such as an immunogen comprising the sequence of amino acids of SEQ ID NOS:28, 29 and/or 30, or the corresponding sequence from a non-USA/Colorado/2013 PEDV isolate.

In additional embodiments, a method of treating or preventing PEDV infection in a porcine subject, or in a piglet born to a female porcine subject, is provided that comprises administering to the porcine subject a therapeutically effective amount of any of the above compositions. In certain embodiments, the porcine subject is a pregnant sow and the composition is administered to a pregnant sow prior to farrowing.

In further embodiments, a method of making a PEDV composition is provided that comprises: (a) inactivating or attenuating a PEDV; and (b) combining the inactivated PEDV with a pharmaceutically acceptable vehicle and an immunological adjuvant selected from (i) alum or (ii) an adjuvant composition comprising a host defense peptide, an immunostimulatory sequence and a polyphosphazine. In certain embodiments, the PEDV is inactivated using beta-propiolactone.

In additional embodiments, a method of making a PEDV composition is provided that comprises; (a) providing at least one isolated immunogen comprising an epitope from a PEDV spike (S) protein, a PEDV ORF3 protein, a PEDV envelope (E) protein, a PEDV membrane (M) protein, and/or a PEDV nucleocapsid (N) protein; and (b) combining the immunogen with a pharmaceutically acceptable vehicle and an immunological adjuvant

In yet further embodiments, an isolated PEDV nucleocapsid immunogen comprising at least one PEDV epitope is provided, wherein the immunogen comprises the sequence of amino acids of SEQ ID NOS:28, 29 and/or 30, or the corresponding sequence from a non-USA/Colorado/2013 PEDV isolate.

In additional embodiments, antibodies specific for a PEDV nucleocapsid immunogen described above are provided, such as polyclonal or monoclonal antibodies, as are compositions comprising the antibodies and a pharmaceutically acceptable vehicle. In further embodiments, methods of making a composition are provided that comprise combining the antibodies with a pharmaceutically acceptable vehicle.

In a further embodiment, a method of detecting PEDV antibodies in a biological sample is provided. The method comprises: (a) reacting the biological sample with an immunogen as described above under conditions which allow PEDV antibodies, when present in the biological sample, to bind to the immunogen to form an antibody/immunogen complex; and (b) detecting the presence or absence of the complex, thereby detecting the presence or absence of PEDV antibodies in the sample.

In an additional embodiment, a method of detecting PEDV infection in a biological sample is provided, wherein the method comprises: (a) reacting the biological sample with antibodies as described above, under conditions which allow PEDV immunogens, when present in the biological sample, to bind to the antibodies to form an antibody/immunogen complex; and (b) detecting the presence or absence of the complex, thereby detecting the presence or absence of PEDV infection in the sample.

In a further embodiment, an immunodiagnostic test kit for detecting PEDV infection is provided, the test kit comprising an immunogen or antibodies as above, and instructions for conducting the immunodiagnostic test.

These and other embodiments of the subject invention will readily occur to those of skill in the art in view of the disclosure herein.

### BRIEF DESCRIPTION OF THE FIGURES

FIGS. 1A-1H (SEQ ID NO:1) show the cDNA genomic sequence for isolate USA/Colorado/2013 PEDV (GenBank Accession no. KF272920). The complete genome includes 28,038 nucleotides (nt), excluding the 3′ poly(A) tail. The genome arrangement and corresponding nucleotide positions are as follows: 5′ untranslated region (UTR), nt 1-292; replicase, nt 293-12646 for 1a, and nt 12601-20637 for 1b; spike (S), nt 20634-24794; open reading frame 3 (ORF3), nt 24794-25468; envelope (E), nt 25449-25679; membrane (M), nt 25687-26367; nucleocapsid (N), nt 26379-27704; and 3′ UTR, nt 27706-28038.

FIG. 2 is an overview of the PEDV challenge model in neonatal pigs as described in the examples.

FIG. 3 is an overview of vaccination trial I as described in the examples.

FIG. 4 shows survival of piglets in trial I as described in the examples.

FIG. 5A and FIG. 5B show clinical symptoms (FIG. 5A) and mortality (FIG. 5B) in piglets after PEDV infection in vaccination trial I as described in the examples.

FIG. 6 is an overview of vaccination trial II as described in the examples.

FIG. 7 shows percent survival of piglets in trial II as described in the examples.

FIG. 8 is a comparison in percent survival between control and alum-adjuvanted vaccine groups as described in the examples.

FIG. 9 shows clinical scores in piglets after PEDV infection in vaccination trial II as described in the examples.

FIG. 10 shows the change in litter weight in piglets after PEDV infection in vaccinated and control piglets in vaccination trial II as described in the examples.

FIG. 11A and FIG. 11B show the individual animal changes in litter weight in vaccinated animals (FIG. 11A) and control animals (FIG. 11B) in vaccination trial II as described in the examples.

FIG. 12 shows the amount of viral shedding in fecal material after PEDV challenge in vaccination trial II as described in the examples. The horizontal bars represent the median values for the group.

FIG. 13 shows fecal score after PEDV challenge in vaccination trial II as described in the examples.

FIG. 14 (SEQ ID NO:23) shows the protein sequence of the Spike (S) protein for isolate USA/Colorado/2013 PEDV (Genbank Accession no. KF272920). The S1 region (amino acids 234-736) and S2 region (amino acids 744-1347) are shown in bold.

FIG. 15 (SEQ ID NO:24) shows the protein sequence of the ORF3 Protein for isolate USA/Colorado/2013 PEDV (Genbank Accession no. KF272920).

FIG. 16 (SEQ ID NO:25) shows the protein sequence of the Envelope Protein (E) for isolate USA/Colorado/2013 PEDV (Genbank Accession no. KF272920).

FIG. 17 (SEQ ID NO:26) shows the protein sequence of the Membrane Protein (M) for isolate USA/Colorado/2013 PEDV (Genbank Accession no. KF272920).

FIG. 18 (SEQ ID NO:27) shows the protein sequence of the Nucleocapsid Protein (N) for isolate USA/Colorado/2013 PEDV (Genbank Accession no. KF272920).

FIG. 19 shows the serum anti-PEDV S1 antibody responses in sows in vaccine trial III. The horizontal bars represent the median of IgG titers of the control (n=3) and vaccinated sows (n=4).

FIG. 20 shows viral neutralization responses in sows in vaccine trial III. The horizontal bars represent the median of IgG titers of the control (n=3) and vaccinated sows (n=4).

FIG. 21 shows colostrum anti-PEDV SI antibody responses in sows in vaccine trial III. The horizontal bar represents the median of IgG titers of the control (n=3) and vaccinated sows (n=4).

FIG. 22 shows the serum anti-PEDV S1 antibody responses in piglets in vaccine trial III. The horizontal bars represent the median of IgG titers of litters from the four vaccinated sows (45 piglets) and from the three control sows (34 piglets).

FIG. 23 shows virus neutralizing antibody titers in sera of piglets in vaccine trial III. The horizontal bar represents the median value.

FIG. 24 shows the weight change in piglets from control sows (●) and piglets from vaccinated sows (▪) in vaccine trial III.

FIG. 25 shows percent survival of piglets in vaccine trial III. Survival curves for piglets from control sows (●) and piglets from vaccinated sows (▪) are shown.

FIG. 26 shows the results of an in vivo evaluation of live PEDV virus infection in neonatal pigs after inactivated vaccine administration.

FIG. 27 shows the immunogenicity of the inactivated vaccine. The horizontal bars represent the median of IgG titers of the various groups.

FIG. 28 shows colostrum anti-PEDV S1 antibody responses in sows in the vaccine field study. The horizontal bar represents the median of IgG titers of the control (n=12) and vaccinated sows (n=12).

FIG. 29 shows the serum anti-PEDV SI antibody responses in piglets in the vaccine field trial. The horizontal bars represent the median of IgG titers of litters from control sows (●) and piglets from vaccinated sows (▪).

FIG. 30 shows percent survival of piglets in the vaccine field trial. Survival curves for piglets from control sows (●) and piglets from vaccinated sows (▪) are shown.

FIG. 31 shows the serum anti-PEDV S1 antibody responses in sows at the time of farrowing in the vaccine field trial. The horizontal bars represent the median of IgG titers of litters from control sows (●) and piglets from vaccinated sows (▪).

FIG. 32 shows the change in litter weight in piglets after PEDV challenge in piglets from control sows (●) and piglets from vaccinated sows (▪) in the vaccine field trial.

FIG. 33 shows the median serum anti-PEDV S1 antibody responses in piglets prior to challenge in the vaccine field trial. Each symbol represents the median value for that litter of piglets (n=18). The horizontal bars represent the median of IgG titers.

FIG. 34 shows the percent survival of piglets at 7 days of age following administration of a control vaccine (Group A); a supernatant PEDV vaccine containing 2×10⁵ viral particles (Group B); a cell pellet PEDV vaccine containing 2×10⁵ viral particles (Group C); and a supernatant PEDV vaccine containing 1×10⁶ viral particles (Group D).

FIG. 35 shows the serum IgG titers in groups of sows administered a control vaccine (Group A); a supernatant PEDV vaccine containing 2×10⁵ viral particles (Group B); a cell pellet PEDV vaccine containing 2×10⁵ viral particles (Group C); and a supernatant PEDV vaccine containing 1×10⁶ viral particles (Group D).

FIG. 36 shows IgG titers from whey in groups of sows administered a control vaccine (Group A); a supernatant PEDV vaccine containing 2×10⁵ viral particles (Group B); a cell pellet PEDV vaccine containing 2×10⁵ viral particles (Group C); and a supernatant PEDV vaccine containing 1×10⁶ viral particles (Group D).

FIG. 37 shows the serum IgG titers of piglets born to groups of sows administered a control vaccine (Group A); a supernatant PEDV vaccine containing 2×10⁵ viral particles (Group B); a cell pellet PEDV vaccine containing 2×10⁵ viral particles (Group C); and a supernatant PEDV vaccine containing 1×10⁶ viral particles (Group D).

### DETAILED DESCRIPTION OF THE INVENTION

The practice of the present invention will employ, unless otherwise indicated, conventional techniques of molecular biology, microbiology, bacteriology, virology, recombinant DNA technology, and immunology, which are within the skill of the art. Such techniques are explained fully in the literature. See, e.g., Sambrook, Fritsch & Maniatis, Molecular Cloning: A Laboratory Manual, current edition; Fundamental Virology, current edition, vol. I & II (B. N. Fields and D. M. Knipe, eds.); DNA Cloning, Vols. I and II (D. N. Glover ed. current edition); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); and Handbook of Experimental Immunology, Vols. I-IV (D. M. Weir and C. C. Blackwell eds., 1986, Blackwell Scientific Publications).

All publications, patents and patent applications cited herein, whether supra or infra, are hereby incorporated by reference in their entirety.

### 1. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a PEDV immunogen” includes a mixture of two or more such immunogens, and the like.

By “PEDV infection” is meant any disorder caused directly or indirectly by PEDV, including without limitation, infection caused by various PEDV strains and isolates. Particular porcine isolates are described in detail below. Such infection includes, without limitation, diarrhea, dehydration, weight loss, viral shedding (e.g., fecal shedding), inappetence, vomiting, rough hair coat, lethargy, morphological differences seen in infected cells such as but not limited to the intestinal villi, and the like.

The term also intends subclinical disease, e.g., where PEDV infection is present but clinical symptoms of disease have not yet been manifested. Subjects with subclinical disease can be asymptomatic but may nonetheless be at risk of developing any of the above disorders, as well as spreading disease by fecal shedding and the like.

The term “polypeptide” when used with reference to a PEDV immunogen, refers to the immunogen, whether native, recombinant or synthetic, which is derived from any PEDV strain or isolate. The polypeptide need not include the full-length amino acid sequence of the reference molecule but can include only so much of the molecule as necessary in order for the polypeptide to retain immunogenicity and/or the ability to treat and/or prevent PEDV infection, as described below. Thus, only one or few epitopes of the reference molecule need be present. Furthermore, the polypeptide may comprise a fusion protein between the full-length reference molecule or a fragment of the reference molecule, and another protein that does not disrupt the reactivity of the PEDV polypeptide. It is readily apparent that the polypeptide may therefore comprise the full-length sequence, fragments, truncated and partial sequences, as well as analogs and precursor forms of the reference molecule. The term also intends deletions, additions and substitutions to the reference sequence, so long as the polypeptide retains immunogenicity.

Thus, the full-length proteins and fragments thereof, as well as proteins with modifications, such as deletions, additions and substitutions (either conservative or non-conservative in nature), to the native sequence, are intended for use herein, so long as the protein maintains the desired activity. These modifications may be deliberate, as through site-directed mutagenesis, or may be accidental, such as through mutations of hosts which produce the proteins or errors due to PCR amplification. Accordingly, active proteins substantially homologous to the parent sequence, e.g., proteins with 70 . . . 80 . . . 85 . . . 90 . . . 95 . . . 98 . . . 99% etc. identity that retain the biological activity, are contemplated for use herein.

The term “peptide” as used herein refers to a fragment of a polypeptide. Thus, a peptide can include a C-terminal deletion, an N-terminal deletion and/or an internal deletion of the native polypeptide, so long as the entire protein sequence is not present. A peptide will generally include at least about 3-10 contiguous amino acid residues of the full-length molecule, and can include at least about 15-25 contiguous amino acid residues of the full-length molecule, or at least about 20-50 or more contiguous amino acid residues of the full-length molecule, or any integer between 3 amino acids and the number of amino acids in the full-length sequence, provided that the peptide in question retains the ability to elicit the desired biological response.

The term “analog” refers to biologically active derivatives of the reference molecule, or fragments of such derivatives, that retain activity, as described above. In general, the term “analog” refers to compounds having a native polypeptide sequence and structure with one or more amino acid additions, substitutions and/or deletions, relative to the native molecule. Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 or 50 conservative or non-conservative amino acid substitutions, or any number between 5-50, so long as the desired function of the molecule remains intact.

A “purified” protein or polypeptide is a protein which is recombinantly or synthetically produced, or isolated from its natural host, such that the amount of the protein of interest present in a composition is substantially higher than that present in a crude preparation. In general, a purified protein will be at least about 50% homogeneous and more preferably at least about 80% to 90% or more homogeneous.

By “biologically active” is meant a PEDV or immunogenic protein derived therefrom, that elicits an immunological response, as defined below, or that is useful in a diagnostic for PEDV disease.

By “subunit vaccine composition” is meant a composition containing at least one immunogen, but not all immunogens, derived from or homologous to an immunogen from PEDV. Such a composition is substantially free of intact virus. Thus, a “subunit vaccine composition” is prepared from at least partially purified (preferably substantially purified) immunogens from PEDV, or recombinant analogs thereof. A subunit vaccine composition can comprise the subunit antigen or antigens of interest substantially free of other antigens or polypeptides from the pathogen. Representative immunogens include those derived from, for example, the spike (S) protein (including those derived from S1 and S2, which include neutralizing epitopes), ORF3, the envelope (E) protein, the membrane (M) protein, and/or the nucleocapsid (N) of PEDV, including the full-length protein or fragments thereof. The sequences of these proteins are known and described in, e.g., GenBank Accession no. KF272920. Moreover, immunogens from multiple isolates or PEDV strains can be present. Also encompassed is the use of consensus sequences from any of the above viral regions based on multiple isolates or strains of PEDV.

By “epitope” is meant a site on an antigen to which specific B cells and T cells respond. The term is also used interchangeably with “antigenic determinant” or “antigenic determinant site.” An epitope can comprise 3 or more amino acids in a spatial conformation unique to the epitope. Generally, an epitope consists of at least 5 such amino acids and, more usually, consists of at least 8-25 such amino acids, such as 10-25 such amino acids or any integer between the stated ranges. The term “epitope” also includes modified sequences of amino acids which stimulate responses which recognize the organism. The epitope can be generated from knowledge of the amino acid and corresponding DNA sequences of the peptide or polypeptide, as well as from the nature of particular amino acids (e.g., size, charge, etc.) and the codon dictionary, without undue experimentation. See, e.g., Ivan Roitt, Essential Immunology, 1988; Kendrew, supra; Janis Kuby, Immunology, 1992 e.g., pp. 79-81.

Methods of determining spatial conformation of amino acids are known in the art and include, for example, x-ray crystallography and 2-dimensional nuclear magnetic resonance. Furthermore, the identification of epitopes in a given protein is readily accomplished using techniques well known in the art, such as by the use of hydrophobicity studies and by site-directed serology. See, also, Geysen et al., Proc. Natl. Acad. Sci. USA (1984) 81:3998-4002 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23:709-715 (technique for identifying peptides with high affinity for a given antibody). Antibodies that recognize the same epitope can be identified in a simple immunoassay showing the ability of one antibody to block the binding of another antibody to a target antigen.

An “immunological response” to a composition or vaccine is the development in the host of a cellular and/or antibody-mediated immune response to the composition or vaccine of interest. Usually, an “immunological response” includes but is not limited to one or more of the following effects: the production of antibodies, such as neutralizing antibodies, B cells, helper T cells, suppressor T cells, and/or cytotoxic T cells and/or gamma delta (γδ) T cells, directed specifically to an antigen or antigens included in the composition or vaccine of interest. Preferably, the host will display a protective immunological response to the PEDV immunogen(s) in question, e.g., the host will be protected from subsequent infection by the pathogen and such protection will be demonstrated by either a reduction or lack of symptoms normally displayed by an infected host or a quicker recovery time.

The terms “immunogenic” PEDV, protein or polypeptide refer to a PEDV, or a protein therefrom which elicits an immunological response as described above. An “immunogenic” protein or polypeptide, as used herein, includes the full-length sequence of the particular PEDV immunogen in question, including any precursor and mature forms, analogs thereof, or immunogenic fragments thereof. By “immunogenic fragment” is meant a fragment of the PEDV immunogen in question which includes one or more epitopes and thus elicits the immunological response described above.

Immunogenic fragments, for purposes of the present invention, will usually be at least about 2 amino acids in length, more preferably about 5 amino acids in length, and most preferably at least about 10 to 15 amino acids in length. There is no critical upper limit to the length of the fragment, which could comprise nearly the full-length of the protein sequence, or even a fusion protein comprising two or more epitopes of the PEDV immunogen in question.

An “antibody” intends a molecule that “recognizes,” i.e., specifically binds to an epitope of interest present in an antigen. By “specifically binds” is meant that the antibody interacts with the epitope in a “lock and key” type of interaction to form a complex between the antigen and antibody, as opposed to non-specific binding that might occur between the antibody and, for instance, components in a mixture that includes the test substance with which the antibody is reacted. Thus, an anti-PEDV antibody is a molecule that specifically binds to an epitope of the PEDV protein in question. The term “antibody” as used herein includes antibodies obtained from both polyclonal and monoclonal preparations, as well as, the following: hybrid (chimeric) antibody molecules (see, for example, Winter et al., Nature (1991) 349:293-299; and U.S. Pat. No. 4,816,567); F(ab′)2 and F(ab) fragments; Fv molecules (non-covalent heterodimers, see, for example, Inbar et al., Proc Natl Acad Sci USA (1972) 69:2659-2662; and Ehrlich et al., Biochem (1980) 19:4091-4096); single-chain Fv molecules (sFv) (see, for example, Huston et al., Proc Natl Acad Sci USA (1988) 85:5879-5883); dimeric and trimeric antibody fragment constructs; minibodies (see, e.g., Pack et al., Biochem (1992) 31:1579-1584; Cumber et al., J Immunology (1992) 149B:120-126); humanized antibody molecules (see, for example, Riechmann et al., Nature (1988) 332:323-327; Verhoeyan et al., Science (1988) 239:1534-1536; and U.K. Patent Publication No. GB 2,276,169, published 21 Sep. 1994); and, any functional fragments obtained from such molecules, wherein such fragments retain immunological binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)₂, Fv, and other fragments, as well as chimeric and humanized homogeneous antibody populations, that exhibit immunological binding properties of the parent monoclonal antibody molecule.

“Homology” refers to the percent identity between two polynucleotide or two polypeptide moieties. Two nucleotide, or two polypeptide sequences are “substantially homologous” to each other when the sequences exhibit at least about 50% , preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified nucleotide or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are well known in the art.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

A “coding sequence” or a sequence which “encodes” a selected polypeptide, is a nucleic acid molecule which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

By “vector” is meant any genetic element, such as a plasmid, phage, transposon, cosmid, chromosome, virus, virion, etc., which is capable of replication when associated with the proper control elements and which can transfer gene sequences to cells. Thus, the term includes cloning and expression vehicles, as well as viral vectors.

By “recombinant vector” is meant a vector that includes a heterologous nucleic acid sequence which is capable of expression in vitro or in vivo.

The term “transfection” is used to refer to the uptake of foreign nucleic acid by a cell, and a cell has been “transfected” when exogenous nucleic acid has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acid moieties into suitable host cells.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A “nucleic acid” sequence refers to a DNA or RNA sequence. The term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

The term DNA or RNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

The term “promoter” is used herein in its ordinary sense to refer to a nucleotide region comprising a regulatory sequence, wherein the regulatory sequence is derived from a gene which is capable of binding a polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

“Operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

For the purpose of describing the relative position of nucleotide sequences in a particular nucleic acid molecule throughout the instant application, such as when a particular nucleotide sequence is described as being situated “upstream,” “downstream,” “3 prime (3′)” or “5 prime (5′)” relative to another sequence, it is to be understood that it is the position of the sequences in the “sense” or “coding” strand of a nucleic acid molecule that is being referred to as is conventional in the art.

As used herein, a “biological sample” refers to a sample of tissue or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells, and cell components.

The terms “effective amount” or “therapeutically effective amount” of a composition or agent, as provided herein, refer to a nontoxic but sufficient amount of the composition or agent to provide the desired “therapeutic effect,” such as to elicit an immune response as described above, preferably preventing, reducing or reversing symptoms associated with PEDV infection.

This effect can be to alter a component of PEDV disease (or disorder) toward a desired outcome or endpoint, such that a subject's disease or disorder shows improvement, often reflected by the amelioration of a sign or symptom relating to the disease or disorder, including without limitation diarrhea, dehydration, weight loss, duration and magnitude of viral shedding (e.g., fecal shedding), inappetence, vomiting, rough hair coat, lethargy, morphological differences seen in infected cells such as but not limited to the intestinal villi, and the like.

A representative therapeutic effect can render the subject negative for PEDV infection when samples from pigs are cultured for PEDV. Similarly, biopsies indicating lowered IgG, IgM and IgA antibody production directed against PEDV can be an indication of a therapeutic effect. Additionally, decreased serum antibodies against PEDV are indicative of a therapeutic effect. As explained above, reduced symptoms of PEDV infection are also indicative of a therapeutic effect.

The exact amount required to produce a therapeutic benefit will vary from subject to subject, depending on the species, age, and general condition of the subject, the severity of the condition being treated, and the particular components of the composition administered, mode of administration, and the like. An appropriate “effective” amount in any individual case may be determined by one of ordinary skill in the art using routine experimentation.

“Treatment” or “treating” PEDV infection includes: (1) preventing PEDV infection, or (2) causing disorders related to PEDV infection to develop or to occur at lower rates in a subject that may be exposed to PEDV, (3) reducing the amount of PEDV present in a subject, and/or reducing the symptoms associated with PEDV infection.

### 2. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

Central to the present invention is the use of PEDV compositions as vaccines to prevent and/or treat PEDV infection. As shown herein, these vaccines can be administered to pregnant sows prior to farrowing, to impart passive immunity to piglets born to the sows. In order to further an understanding of the invention, a more detailed discussion is provided below regarding PEDV, PEDV formulations, as well as various uses thereof.

PEDV

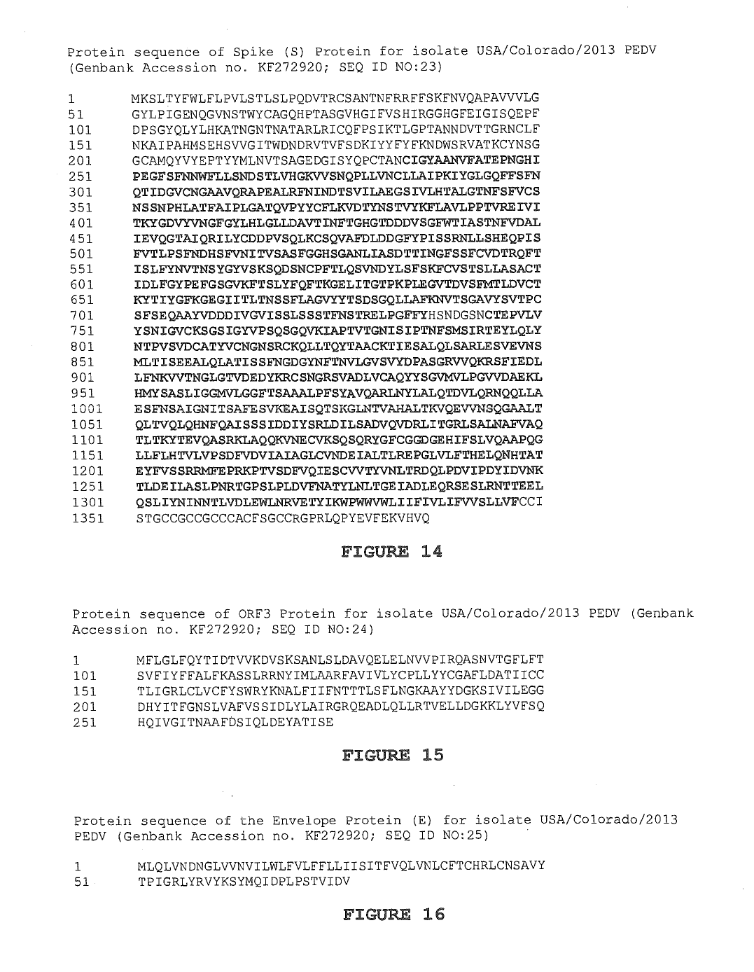

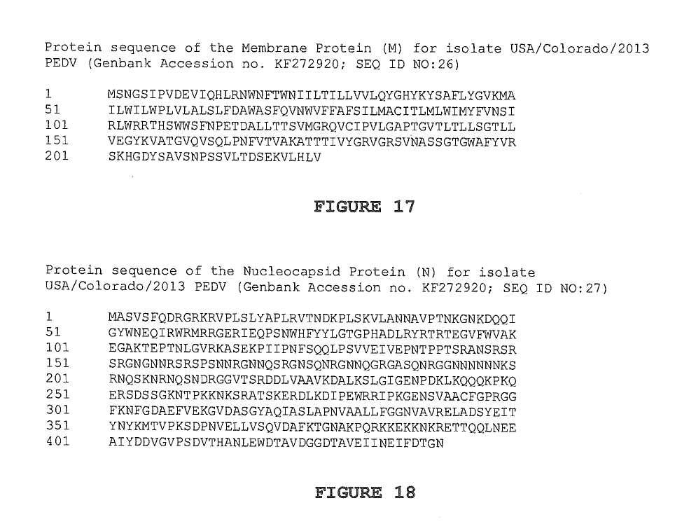

PEDV for use in vaccines, as well as other immunogenic compositions for therapy or diagnosis, can include attenuated or inactivated virus, as well as subunit compositions, including isolated PEDV immunogens, such as immunogens derived from any of the various regions of the PEDV genome. Representative immunogens include those derived from, for example, the spike (S) protein, such as from S1 and/or S2, ORF3, the envelope (E) protein, the membrane (M) protein, and/or the nucleocapsid (N) of PEDV, including the full-length protein, fragments thereof or fusions thereof. The sequences of these proteins are known and described in, e.g., GenBank Accession no. KF272920. Representative S, ORF3, E, M and N proteins from isolate USA/Colorado/2013 PEDV are shown in FIGS. 14-18 (SEQ ID NOS:23-27), respectively. It is to be understood that the corresponding proteins and immunogenic fragments thereof, from other non-USA/Colorado/2013 PEDV isolates will also be of use herein. Such corresponding proteins can easily be determined by aligning the amino acid sequences from different isolates and comparing the sequences with USA/Colorado/2013 PEDV to determine regions of homology.

The proteins for use in the subject compositions can include epitopes present in these regions, the full-length region or portions thereof. In this regard, at least three epitopes in the N region have been discovered by the inventors herein. These epitopes occur in the N protein at amino acid positions 285-304 (PKGENSVAACFGPRGGFKNF, SEQ ID NO:28); amino acid positions 257-280 (GKNTPKKNKSRATSKERDLKDIPE, SEQ ID NO:29); and 393-412 (TTQQLNEEAIYDDVGVPSDV, SEQ ID NO:30), all numbered relative to SEQ ID NO:27 (N protein of isolate USA/Colorado/2013 PEDV; GenBank Accession no. KF272920). Proteins or peptides including these epitopes, as well as corresponding epitopes in the S, ORF3, E, M and/or N proteins in other isolates will find use herein.

Moreover, immunogens from multiple isolates or PEDV strains can be present. Proteins including consensus sequences derived from multiple strains or isotypes can also be used. The various proteins can be present individually in a composition, or may be present in a multiple epitope fusion protein. Additionally, the peptides of the invention may include fusions of more than one PEDV protein or peptide and the fusions may include the molecules present as linear repeats, in the same orientation, i.e., the C-terminal amino acid of the first protein or peptide is fused to the N-terminal amino acid of the repeat of the protein, the C-terminal amino acid of this repeat is fused to the N-terminal amino acid of the next repeat, etc. Alternatively, one or more of the repeats can be present in an inverted orientation, i.e., the C-terminal amino acid of the first PEDV molecule is fused to the C-terminal amino acid of the repeat of the PEDV molecule, etc.

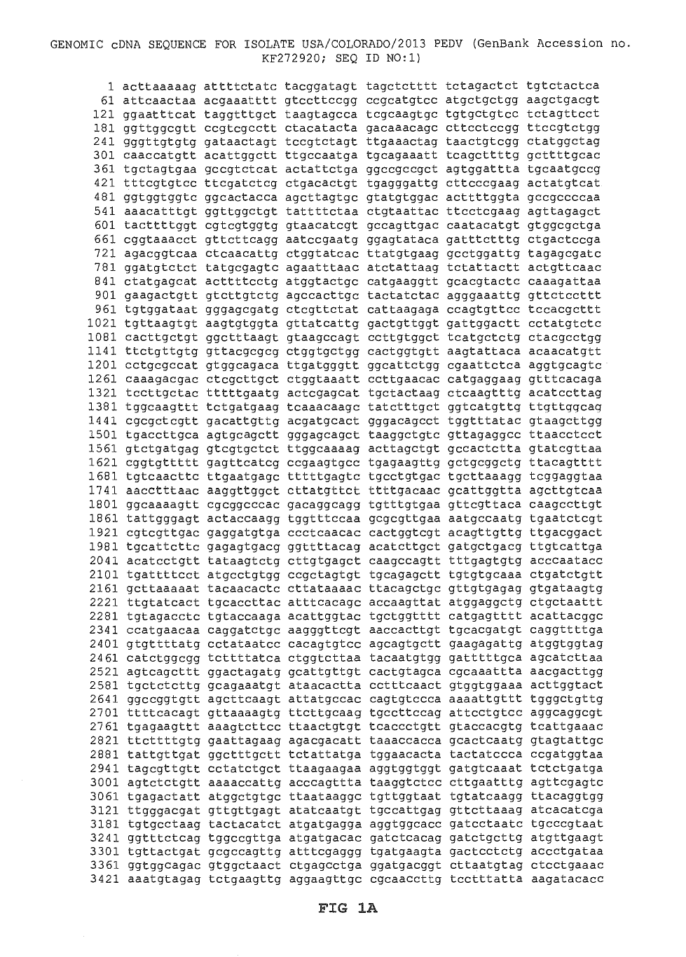

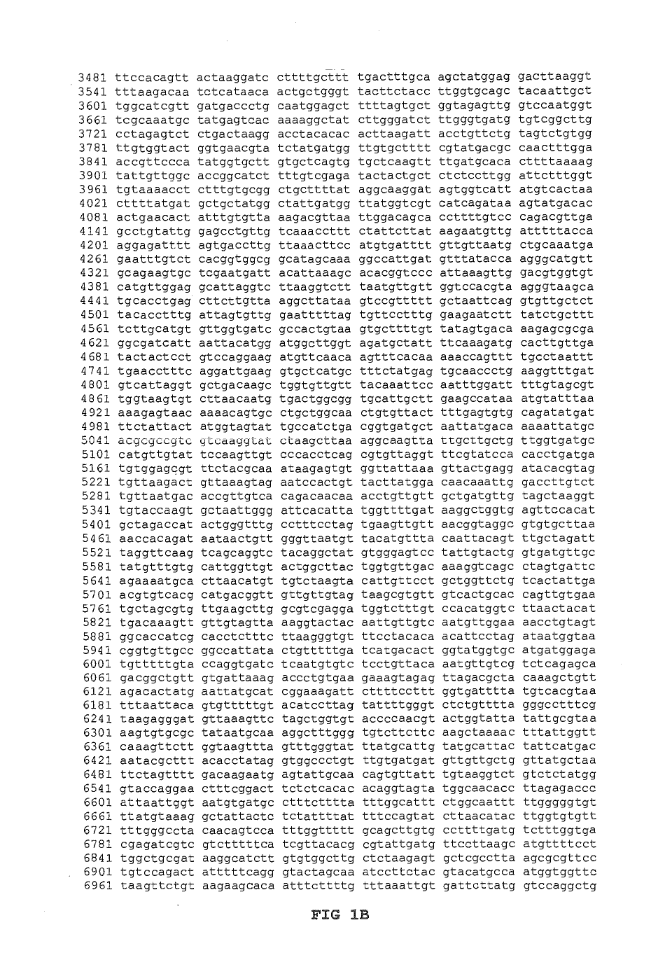

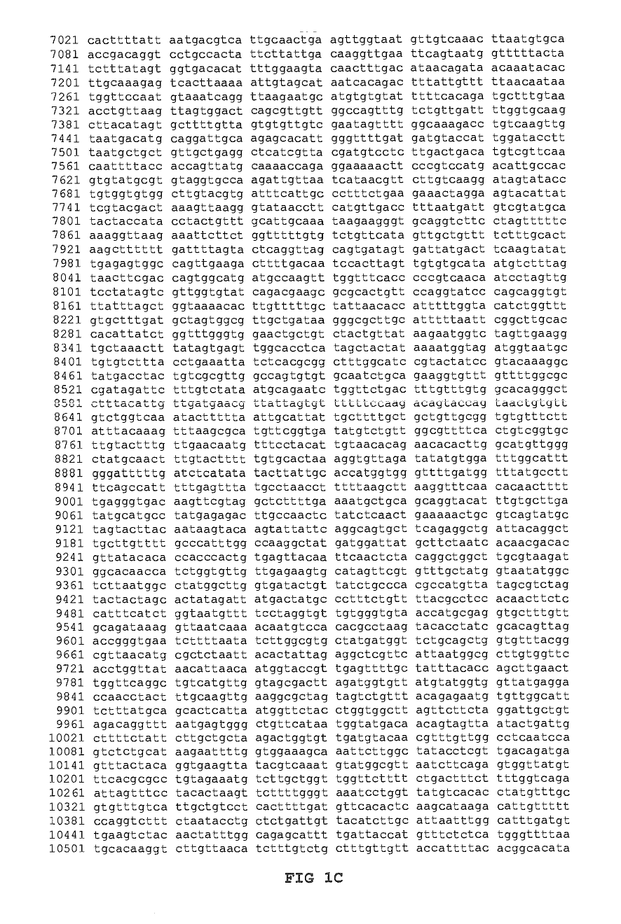

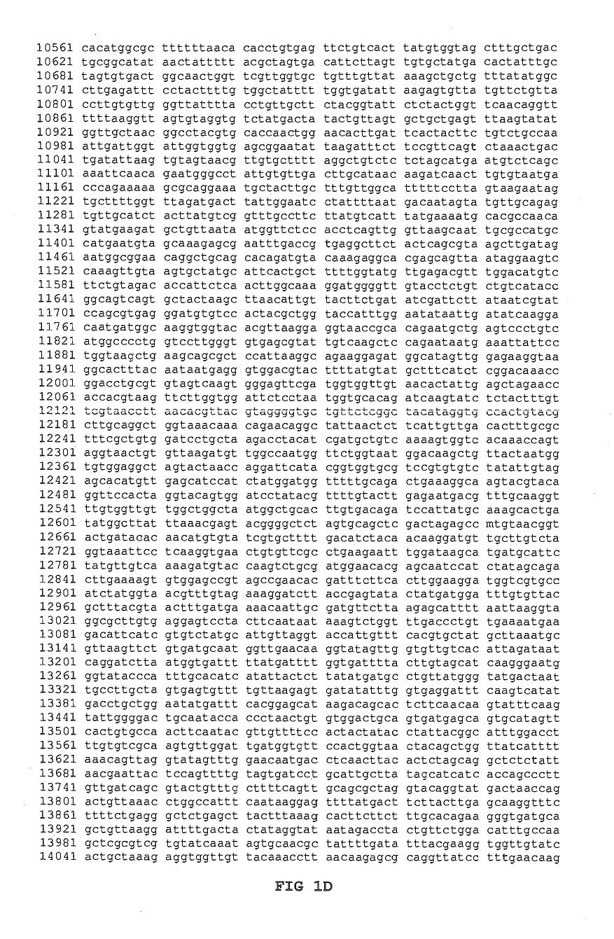

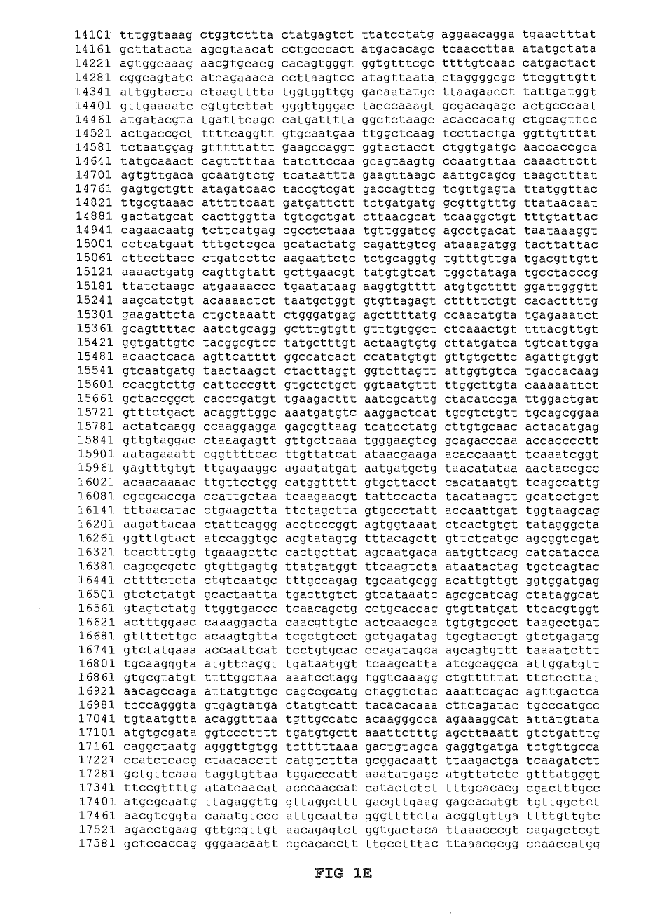

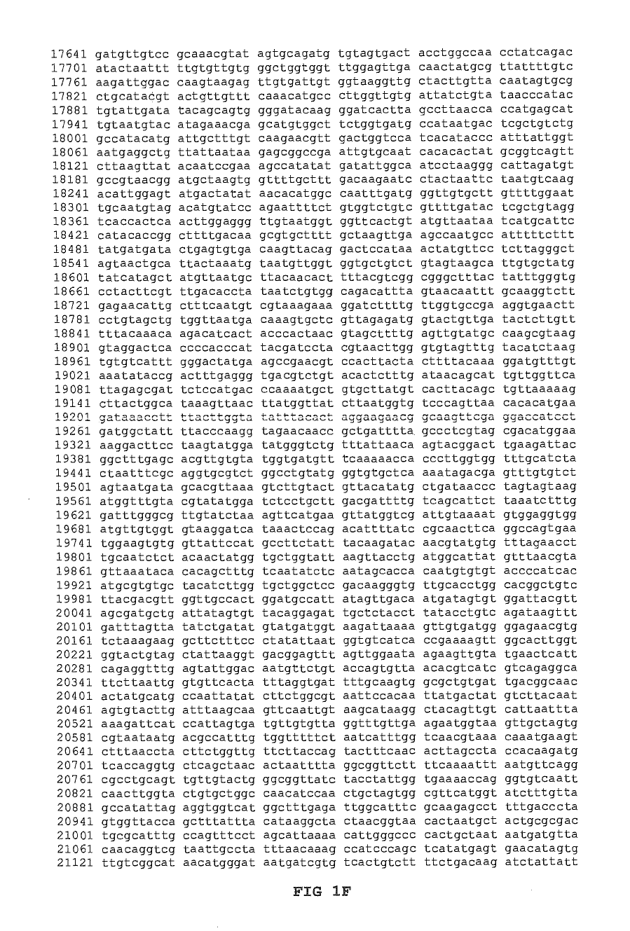

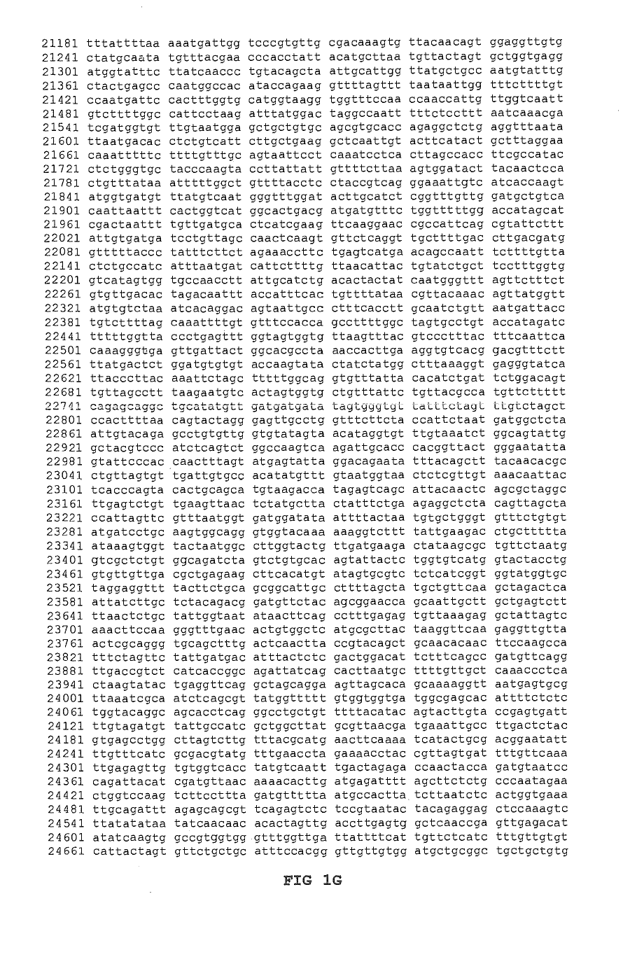

PEDV and immunogens therefrom for use in compositions may be derived from any PEDV strain and isolate. A large number of PEDVs are known. The genomic sequences of these isolates, including the sequences for the various regions of the virus are known, for example strain USA/Colorado/20I3 (GenBank: KF272920.1, FIGS. 1A-1H); strain: Tottori2/JPN/2014 (GenBank: LC022792.1); strain CV777 (GenBank: AF353511.1); strain FR/001/2014 (GenBank: KR011756.1); strain MEX/104/2013 (GenBank: KJ645708.1); strain USA/Minnesota84/2013 (GenBank: KJ645707.1); strain USA/Minnesota71/2013 (GenBank: KJ645706.1); strain USA/Minnesota61/2013 (GenBank: KJ645705.1); strain USA/Minnesota52/2013 (GenBank: KJ645704.1); strain USA/Minnesota127/2014 (GenBank: KJ645703.1); strain USA/Ohio126/2014 (GenBank: KJ645702.1); strain USA/Kansas125/2014 (GenBank: KJ645701.1); strain MEX/124/2014 (GenBank: KJ645700.1); strain USA/Ohio123/2014 (GenBank: KJ645699.1); strain USA/Ohio120/2014 (GenBank: KJ645698.1); strain USA/Texas128/2013 (GenBank: KJ645697.1); strain USA/Iowa107/2013 (GenBank: KJ645696.1); strain USA/Iowa106/2013 (GenBank: KJ645695.1); strain USA/Iowa103/2013 (GenBank: KJ645694.1); strain USA/Missouri102/2013 (GenBank: KJ645693.1); strain USA/Missouri101/2013 (GenBank: KJ645692.1); strain USA/Minnesota100/2013 (GenBank: KJ645691.1); strain USA/Illinois98/2013 (GenBank: KJ645690.1); strain USA/Illinois97/2013 (GenBank: KJ645689.1); strain USA/Iowa96/2013 (GenBank: KJ645688.1); strain USA/Minnesota95/2013 (GenBank: KJ645687.1); strain USA/Minnesota94/2013 (GenBank: KJ645686.1); USA/Missouri93/2013 (GenBank: KJ645685.1); strain USA/Missouri92/2013 (GenBank: KJ645684.1); strain USA/NorthCarolina91/2013 (GenBank: KJ645683.1); strain USA/Minnesota90/2013 (GenBank: KJ645682.1); strain USA/Tennessee56/2013 (GenBank: KJ645654.1); strain USA/Wisconsin55/2013 (GenBank: KJ645653.1); strain USA/Colorado47/2013 (GenBank: KJ645651.1); strain USA/Oklahoma38/2013 (GenBank: KJ645644.1); strain USA/Colorado30/2013 (GenBank: KJ645638.1); strain PEDV-WS (GenBank: KM609213.1); strain PEDV-LYG (GenBank: KM609212.1); strain PEDV-LS (GenBank: KM609211.1); strain PEDV-LY (GenBank: KM609210.1); strain PEDV-CHZ (GenBank: KM609209.1); strain PEDV-15F (GenBank: KM609208.1); strain PEDV-14 (GenBank: KM609207.1); strain PEDV-10F (GenBank: KM609206.1); strain PEDV-8C (GenBank: KM609205.1); strain PEDV-7C (GenBank: KM609204.1); strain PEDV-1C (GenBank: KM609203.1).

PEDV immunogens, including whole PEDV virus, can be produced using a variety of techniques. For example, PEDV and immunogens therefrom can be obtained directly from PEDV-infected subjects, such as swine, using techniques well known in the art. PEDV RNA and DNA can be obtained using polymerase chain reaction (PCR) techniques, using methods well known in the art, such as RT-PCT.

PEDV so obtained can be replicated in various cell lines, such as African green monkey kidney (Vero) cells (see, e.g., Crawford et al., Vet. Res (2015) 46:49, as well as the examples herein), such as Vero 76 cells; duck intestinal epithelial cells (MK-DIEC) (Khatri, M., Emerging Infectious Dis. (2015) Volume 21); porcine kidney cells; MDCK cells; etc. Culture conditions for the above cell types are described in a variety of publications. The cell culture conditions to be used for the desired application (temperature, cell density, pH value, etc.) are variable over a very wide range depending on the cell line employed and can readily be adapted to the requirements of the PEDV virus in question. Methods for propagating PEDV in cultured cells include the steps of inoculating the cultured cells with PEDV, cultivating the infected cells for a desired time period for virus propagation, such as for example as determined by virus titer or virus antigen expression (e.g., between 24 and 168 hours after inoculation) and collecting the propagated virus. The cultured cells are inoculated with the virus at a desired multiplicity of infection (MOI), readily determined by one of skill in the art. The infected cell culture (e.g., monolayers) may be removed either by freeze-thawing or by enzymatic action to increase the viral content of the harvested culture supernatants. The harvested fluids are then either inactivated or stored frozen.

Methods of inactivating or killing viruses are known in the art. Such methods destroy the ability of the viruses to infect mammalian cells. Inactivation can be achieved using either chemical or physical means. Chemical means for inactivating PEDV include treatment of the virus with an effective amount of one or more of the following agents: detergents, formaldehyde, formalin, ß-propiolactone, or UV light. Other methods of viral inactivation are known in the art, such as for example binary ethylamine, acetyl ethyleneimine, or gamma irradiation.

For example, ß-propiolactone may be used at concentrations such as 0.005% to 0.5%, such as 0.01% to 0.3%, for example 0.03% to 0.2%, e.g., 0.05% to 0.1%, and any percentage between the stated ranges. The inactivating agent is added to virus-containing cultures (virus material) prior to or after harvesting. The cultures can be used directly or cells disrupted to release cell-associated virus prior to harvesting. Further, the inactivating agent may be added after cultures have been stored frozen and thawed, or after one or more steps of purification to remove cell contaminants. ß-propiolactone is added to the virus material, with the adverse shift in pH to acidity being controlled with a base, such as sodium hydroxide (e.g., 1 N NaOH) or sodium bicarbonate solution. The combined inactivating agent-virus materials are incubated at temperatures from 4° C. to 37° C., for incubation times of preferably 1 hour to 72 hours, such as 2 hours to 24 hours, e.g., 5 hours to 20 hours, 8 hours to 18 hours, or any time period within the stated ranges.

Alternatively, binary ethyleneimine (BEI) can be used to inactivate virus. One representative method of inactivating PEDV is as follows. BEI is made by mixing equal volumes of a 0.2 molar bromoethylamine hydrobromide solution with a 0.4 molar sodium hydroxide solution. The mixture is incubated at about 37° C. for 60 minutes. The resulting cyclized inactivant, BEI, is added to the virus materials at 0.5 to 4 percent, and preferably at 1 to 3 percent, volume to volume. The inactivating virus materials are held from about 4° C. to 37° C. for 24 to 72 hours with periodic agitation. At the end of this incubation, 20 ml of a sterile 1 molar sodium thiosulfate solution is added to insure neutralization of the BEI. Diluted and undiluted samples of the inactivated virus materials are added to susceptible cell (tissue) culture to detect any non-inactivated virus.

The cultured cells are passaged multiple times and examined for the presence of PEDV based on any of a variety of methods, such as, for example, cytopathic effect (CPE) and antigen detection. Such tests allow determination of complete virus inactivation.

Methods of purification of inactivated virus are known in the art and may include one or more of gradient centrifugation, ultracentrifugation, continuous-flow ultracentrifugation and chromatography, such as ion exchange chromatography, size exclusion chromatography, and liquid affinity chromatography. Other examples of purification methods suitable for use in the invention include polyethylene glycol or ammonium sulfate precipitation, as well as ultrafiltration and microfiltration.

The purified viral preparation is substantially free of contaminating proteins derived from the cells or cell culture and preferably comprises less than about 50 pg cellular nucleic acid/μg virus antigen. Still more preferably, the purified viral preparation comprises less than about 20 pg, and even more preferably, less than about 10 pg. Methods of measuring host cell nucleic acid levels in a viral sample are known in the art. Standardized methods approved or recommended by regulatory authorities such as the WHO or the FDA are preferred. Other assays include PCR detection of PEDV in tissue culture and in vivo virus detection assays as described in the examples herein.

The invention also includes compositions comprising attenuated PEDV. As used herein, attenuation refers to the decreased virulence of PEDV in a porcine subject. Methods of attenuating viruses are known in the art. Such methods include serial passage of the virus in cultured cells as described above, until the virus demonstrates attenuated function. The temperature at which the virus is grown can be any temperature at which tissue culture passage attenuation occurs. Attenuated function of the virus after one or more passages in cell culture can be measured by one skilled in the art. Evidence of attenuated function may be indicated by decreased levels of viral replication or by decreased virulence in an animal model, as described above.

One particular method of producing an attenuated PEDV includes passage of the virus in cell culture at suboptimal or “cold” temperatures and/or introduction of attenuating mutations into the PEDV genome by random mutagenesis (e.g., chemical mutagenesis using for example 5-fluorouracil) or site specific-directed mutagenesis. Cold adaptation generally includes passage at temperatures between about 20° C. to about 32° C., such as between temperatures of about 22° C. to about 30° C., e.g., between temperatures of about 24° C. and 28° C. The cold adaptation or attenuation may be performed by passage at increasingly reduced temperatures to introduce additional growth restriction mutations. The number of passages required to obtain safe, immunizing attenuated virus is dependent at least in part on the conditions employed. Periodic testing of the PEDV culture for virulence and immunizing ability in animals can be used to readily determine the parameters for a particular combination of tissue culture and temperature.

PEDV can also be attenuated by mutating one or more of the various viral regions, as described above, to reduce expression of the viral structural or nonstructural proteins. The attenuated PEDV may comprise one or more additions, deletions or insertion in one or more of the regions of the viral genome. For example, epitopes from any of the viral regions can be mutated in order to reduce virulence of the PEDV in question, including mutations of epitopes in the spike (S) protein, including S1 and/or S2, ORF3, the envelope (E) protein, the membrane (M) protein, and/or the nucleocapsid (N) of PEDV. In this regard, at least three epitopes in the N region have been discovered by the inventors herein and these epitopes can be mutated to produce attenuated PEDV strains. These epitopes occur in the N protein at amino acid positions 285-304 (PKGENSVAACFGPRGGFKNF, SEQ ID NO:28); amino acid positions 257-280 (GKNTPKKNKSRATSKERDLKDIPE, SEQ ID NO:29); and 393-412 (TTQQLNEEAIYDDVGVPSDV, SEQ ID NO:30), all numbered relative to SEQ ID NO:27 (N protein of isolate USA/Colorado/2013 PEDV; GenBank Accession no. KF272920). It is to be understood that the corresponding epitopes, from other PEDV isolates can also be mutated in order to produce an attenuated virus. Such corresponding epitopes can easily be determined by aligning the amino acid sequences from different isolates and comparing the sequences with USA/Colorado/2013 PEDV to determine regions of homology.

Once attenuated, the virus is purified using techniques known in the art, such as described above with reference to inactivated viruses.

Subunit compositions can also be produced. For example, the subunit compositions can comprise one or more immunogens derived from any of the viral genomic regions as described herein, such as but not limited to immunogens comprising one or more of SEQ ID NOS:28, 29 and 30. The compositions can be generated using recombinant methods, well known in the art. In this regard, oligonucleotide probes can be devised based on the sequence of the PEDV genome and used to probe genomic or cDNA libraries for PEDV genes encoding for the immunogens useful in the present invention. The genes can then be further isolated using standard techniques and, if desired, restriction enzymes employed to mutate the gene at desired portions of the full-length sequence. Alternatively, nucleic acid sequences encoding the proteins of interest can be prepared synthetically rather than cloned. The sequences can be designed with the appropriate codons for the particular amino acid sequence. In general, one will select preferred codons for the intended host if the sequence will be used for expression. The complete sequence is assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311. PEDV genes can also be isolated directly from viruses using known techniques, such as phenol extraction, and the sequence can be further manipulated to produce any desired alterations. See, e.g., Sambrook et al., supra, for a description of techniques used to obtain and isolate DNA.

Once coding sequences for the desired proteins have been prepared or isolated, they can be cloned into any suitable vector or replicon. Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

The gene can be placed under the control of a promoter, ribosome binding site (for bacterial expression) and, optionally, an operator (collectively referred to herein as “control” elements), so that the DNA sequence encoding the desired protein is transcribed into RNA in the host cell transformed by a vector containing this expression construction. The coding sequence may or may not contain a signal peptide or leader sequence. If signal sequences are included, they can either be the native, homologous sequences, or heterologous sequences. Leader sequences can be removed by the host in post-translational processing. See, e.g., U.S. Pat. Nos. 4,431,739; 4,425,437; 4,338,397.

Other regulatory sequences may also be desirable which allow for regulation of expression of the protein sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

In some cases it may be necessary to modify the coding sequence so that it may be attached to the control sequences with the appropriate orientation; i.e., to maintain the proper reading frame. It may also be desirable to produce mutants or analogs of the sequence of interest. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are described in, e.g., Sambrook et al., supra; DNA Cloning, Vols. I and II, supra; Nucleic Acid Hybridization, supra.

It is often desirable that the polypeptides prepared using the above systems are fusion polypeptides. As with nonfusion proteins, these proteins may be expressed intracellularly or may be secreted from the cell into the growth medium. Furthermore, plasmids can be constructed which include a chimeric gene sequence, encoding e.g., multiple PEDV immunogens. The gene sequences can be present in a dicistronic gene configuration. Additional control elements can be situated between the various genes for efficient translation of RNA from the distal coding region. Alternatively, a chimeric transcription unit having a single open reading frame encoding the multiple antigens can also be constructed. Either a fusion can be made to allow for the synthesis of a chimeric protein or alternatively, protein processing signals can be engineered to provide cleavage by a protease such as a signal peptidase, thus allowing liberation of the two or more proteins derived from translation of the template RNA. The processing protease may also be expressed in this system either independently or as part of a chimera with the antigen and/or cytokine coding region(s). The protease itself can be both a processing enzyme and a vaccine antigen.

The expression vector is then used to transform an appropriate host cell. The molecules can be expressed in a wide variety of systems, including insect, mammalian, bacterial, viral and yeast expression systems, all well known in the art. For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego, Calif. (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney cells (e.g., HEK 293), human hepatocellular carcinoma cells (e.g., Hep G2), Madin-Darby bovine kidney (“MDBK”) cells, as well as others. Similarly, bacterial hosts such as E. coli, Bacillus subtilis , and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the immunogens of the present invention are produced by growing host cells transformed by an expression vector under conditions whereby the immunogen of interest is expressed. The immunogen is then isolated from the host cells and purified. If the expression system provides for secretion of the immunogen, the immunogen can be purified directly from the media. If the immunogen is not secreted, it is isolated from cell lysates. The selection of the appropriate growth conditions and recovery methods are within the skill of the art.

The PEDV immunogens may also be produced by chemical synthesis such as by solid phase or solution peptide synthesis, using methods known to those skilled in the art. Chemical synthesis of peptides may be preferable if the antigen in question is relatively small. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis.

PEDV Antibodies

The PEDV immunogens of the present invention can be used to produce antibodies for therapeutic, diagnostic and purification purposes. These antibodies may be polyclonal or monoclonal antibody preparations, monospecific antisera, human antibodies, or may be hybrid or chimeric antibodies, such as humanized antibodies, altered antibodies, F(ab′)2 fragments, F(ab) fragments, Fv fragments, single-domain antibodies, dimeric or trimeric antibody fragment constructs, minibodies, or functional fragments thereof which bind to the antigen in question. Antibodies are produced using techniques well known to those of skill in the art and disclosed in, for example, U.S. Pat. Nos. 4,011,308; 4,722,890; 4,016,043; 3,876,504; 3,770,380; and 4,372,745.

For example, the PEDV molecules can be used to produce PEDV-specific polyclonal and monoclonal antibodies for use in diagnostic and detection assays, for purification and for use as therapeutics, such as for passive immunization. Such polyclonal and monoclonal antibodies specifically bind to the PEDV molecules in question. In particular, the PEDV proteins can be used to produce polyclonal antibodies by administering the protein to a mammal, such as a mouse, a rat, a rabbit, a goat, or a horse. Serum from the immunized animal is collected and the antibodies are purified from the plasma by, for example, precipitation with ammonium sulfate, followed by chromatography, preferably affinity chromatography. Techniques for producing and processing polyclonal antisera are known in the art.

Mouse and/or rabbit monoclonal antibodies directed against epitopes present in the PEDV protein can also be readily produced. In order to produce such monoclonal antibodies, the mammal of interest, such as a rabbit or mouse, is immunized, such as by mixing or emulsifying the antigen in saline, preferably in an adjuvant such as Freund's complete adjuvant (“FCA”), and injecting the mixture or emulsion parenterally (generally subcutaneously or intramuscularly). The animal is generally boosted 2-6 weeks later with one or more injections of the antigen in saline, preferably using Freund's incomplete adjuvant (“FIA”).

Antibodies may also be generated by in vitro immunization, using methods known in the art. See, e.g., James et al., J. Immunol. Meth. (1987) 100:5-40.

Polyclonal antisera is then obtained from the immunized animal. However, rather than bleeding the animal to extract serum, the spleen (and optionally several large lymph nodes) is removed and dissociated into single cells. If desired, the spleen cells (splenocytes) may be screened (after removal of nonspecifically adherent cells) by applying a cell suspension to a plate or well coated with the antigen. B-cells, expressing membrane-bound immunoglobulin specific for the antigen, will bind to the plate, and are not rinsed away with the rest of the suspension. Resulting B-cells, or all dissociated splenocytes, are then induced to fuse with cells from an immortalized cell line (also termed a “fusion partner”), to form hybridomas. Typically, the fusion partner includes a property that allows selection of the resulting hybridomas using specific media. For example, fusion partners can be hypoxanthine/aminopterin/thymidine (HAT)-sensitive.

If rabbit-rabbit hybridomas are desired, the immortalized cell line will be from a rabbit. Such rabbit-derived fusion partners are known in the art and include, for example, cells of lymphoid origin, such as cells from a rabbit plasmacytoma as described in Spieker-Polet et al., Proc. Natl. Acad. Sci. USA (1995) 92:9348-9352 and U.S. Pat. No. 5,675,063, or the TP-3 fusion partner described in U.S. Pat. No. 4,859,595, incorporated herein by reference in their entireties. If a rabbit-mouse hybridoma or a rat-mouse or mouse-mouse hybridoma, or the like, is desired, the mouse fusion partner will be derived from an immortalized cell line from a mouse, such as a cell of lymphoid origin, typically from a mouse myeloma cell line. A number of such cell lines are known in the art and are available from the ATCC.

Fusion is accomplished using techniques well known in the art. Chemicals that promote fusion are commonly referred to as fusogens. These agents are extremely hydrophilic and facilitate membrane contact. One particularly preferred method of cell fusion uses polyethylene glycol (PEG). Another method of cell fusion is electrofusion. In this method, cells are exposed to a predetermined electrical discharge that alters the cell membrane potential. Additional methods for cell fusion include bridged-fusion methods. In this method, the antigen is biotinylated and the fusion partner is avidinylated. When the cells are added together, an antigen-reactive B cell-antigen-biotin-avidin-fusion partner bridge is formed. This permits the specific fusion of an antigen-reactive cell with an immortalizing cell. The method may additionally employ chemical or electrical means to facilitate cell fusion.

Following fusion, the cells are cultured in a selective medium (e.g., HAT medium). In order to enhance antibody secretion, an agent that has secretory stimulating effects can optionally be used, such as IL-6. See, e.g., Liguori et al., Hybridoma (2001) 20:189-198. The resulting hybridomas can be plated by limiting dilution, and are assayed for the production of antibodies which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (e.g., as ascites in mice). For example, hybridomas producing PEDV-specific antibodies can be identified using RIA or ELISA and isolated by cloning in semi-solid agar or by limiting dilution. Clones producing the desired antibodies can be isolated by another round of screening.

An alternative technique for generating monoclonal antibodies is the selected lymphocyte antibody method (SLAM). This method involves identifying a single lymphocyte that is producing an antibody with the desired specificity or function within a large population of lymphoid cells. The genetic information that encodes the specificity of the antibody (i.e., the immunoglobulin V_{H} and V_{L} DNA) is then rescued and cloned. See, e.g., Babcook et al., Proc. Natl. Acad. Sci. USA (1996) 93:7843-7848, for a description of this method.

For further descriptions of rabbit monoclonal antibodies and methods of making the same from rabbit-rabbit and rabbit-mouse fusions, see, e.g., U.S. Pat. No. 5,675,063 (rabbit-rabbit); U.S. Pat. No. 4,859,595 (rabbit-rabbit); U.S. Pat. No. 5,472,868 (rabbit-mouse); and U.S. Pat. No. 4,977,081 (rabbit-mouse). For a description of the production of conventional mouse monoclonal antibodies, see, e.g., Kohler and Milstein, Nature (1975) 256:495-497.

It may be desirable to provide chimeric antibodies. By “chimeric antibodies” is intended antibodies that are preferably derived using recombinant techniques and which comprise both human (including immunologically “related” species, e.g., chimpanzee) and non-human components. Such antibodies are also termed “humanized antibodies.” Preferably, humanized antibodies contain minimal sequence derived from non-human immunoglobulin sequences. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. See, for example, U.S. Pat. Nos. 5,225,539; 5,585,089; 5,693,761; 5,693,762; 5,859,205. In some instances, framework residues of the human immunoglobulin are replaced by corresponding non-human residues (see, for example, U.S. Pat. Nos. 5,585,089; 5,693,761; 5,693,762). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al., Nature (1986) 331:522-525; Riechmann et al., Nature (1988) 332:323-329; and Presta, Curr. Op. Struct. Biol. (1992) 2:593-596.

Also encompassed are xenogeneic or modified antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. No. 5,939,598, incorporated herein by reference in its entirety.

Antibody fragments which retain the ability to recognize the peptide of interest, will also find use herein. A number of antibody fragments are known in the art which comprise antigen-binding sites capable of exhibiting immunological binding properties of an intact antibody molecule. For example, functional antibody fragments can be produced by cleaving a constant region, not responsible for antigen binding, from the antibody molecule, using e.g., pepsin, to produce F(ab′)2 fragments. These fragments will contain two antigen binding sites, but lack a portion of the constant region from each of the heavy chains. Similarly, if desired, Fab fragments, comprising a single antigen binding site, can be produced, e.g., by digestion of polyclonal or monoclonal antibodies with papain. Functional fragments, including only the variable regions of the heavy and light chains, can also be produced, using standard techniques such as recombinant production or preferential proteolytic cleavage of immunoglobulin molecules. These fragments are known as FV. See, e.g., Inbar et al., Proc. Nat. Acad. Sci. USA (1972) 69:2659-2662; Hochman et al., Biochem. (1976) 15:2706-2710; and Ehrlich et al., Biochem. (1980) 19:4091-4096.

A phage-display system can be used to expand antibody molecule populations in vitro. Saiki, et al., Nature (1986) 324:163; Scharf et al., Science (1986) 233:1076; U.S. Pat. Nos. 4,683,195 and 4,683,202; Yang et al., J Mol Biol. (1995) 254:392; Barbas, III et al., Methods: Comp. Meth Enzymol. (1995) 8:94; Barbas, III et al., Proc Natl Acad Sci USA (1991) 88:7978.

Once generated, the phage display library can be used to improve the immunological binding affinity of the Fab molecules using known techniques. See, e.g., Figini et al., J. Mol. Biol. (1994) 239:68. The coding sequences for the heavy and light chain portions of the Fab molecules selected from the phage display library can be isolated or synthesized, and cloned into any suitable vector or replicon for expression. Any suitable expression system can be used, including those described above.

Single chain antibodies can also be produced. A single-chain Fv (“sFv” or “scFv”) polypeptide is a covalently linked V_{H}-V_{L} heterodimer which is expressed from a gene fusion including V_{H}- and V_{L}-encoding genes linked by a peptide-encoding linker. Huston et al., Proc. Nat. Acad. Sci. USA (1988) 85:5879-5883. A number of methods have been described to discern and develop chemical structures (linkers) for converting the naturally aggregated, but chemically separated, light and heavy polypeptide chains from an antibody V region into an sFv molecule which will fold into a three dimensional structure substantially similar to the structure of an antigen-binding site. See, e.g., U.S. Pat. Nos. 5,091,513, 5,132,405 and 4,946,778, incorporated herein by reference in their entireties. The sFv molecules may be produced using methods described in the art. See, e.g., Huston et al., Proc. Nat. Acad. Sci. USA (1988) 85:5879-5883; U.S. Pat. Nos. 5,091,513, 5,132,405 and 4,946,778. Design criteria include determining the appropriate length to span the distance between the C-terminus of one chain and the N-terminus of the other, wherein the linker is generally formed from small hydrophilic amino acid residues that do not tend to coil or form secondary structures. Such methods have been described in the art. See, e.g., U.S. Pat. Nos. 5,091,513, 5,132,405 and 4,946,778. Suitable linkers generally comprise polypeptide chains of alternating sets of glycine and serine residues, and may include glutamic acid and lysine residues inserted to enhance solubility.

“Mini-antibodies” or “minibodies” will also find use with the present compositions. Minibodies are sFv polypeptide chains which include oligomerization domains at their C-termini, separated from the sFv by a hinge region. Pack et al., Biochem. (1992) 31:1579-1584. The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al., Biochem. (1992) 31:1579-1584; Cumber et al., J. Immunology (1992) 149B:120-126.

Polynucleotide sequences encoding the antibodies and immunoreactive fragments thereof, described above, are readily obtained using standard techniques, well known in the art, such as those techniques described above with respect to the recombinant production of the PEDV molecules.

An anti-PEDV antibody may have therapeutic benefit and can be used to confer passive immunity to the subject in question. Alternatively, antibodies can be used in diagnostic applications, described further below, as well as for purification of the PEDV molecules.

PEDV Formulations and Administration

The inactivated, attenuated or isolated PEDV immunogens of the present invention can be formulated into compositions, such as vaccine compositions, either alone or in combination with other antigens, for use in immunizing subjects as described below. For example, the compositions can include additional immunogens from pathogens that cause disease in pigs, such as but not limited to, immunogens from porcine parvovirus, porcine circovirus, porcine reproductive and respiratory syndrome virus, swine influenza, pseudorabies virus, pestivirus which causes porcine swine fever, porcine lymphotropic herpesviruses (PLHV1 and PLHV2), Mycoplasma spp, Helicobacter spp, Campylobacter spp, Lawsonia spp, Actinobacillus pleuropneumoniae, Haemophilus parasuis, Streptococcus spp, Pasteurella spp, Salmonella spp, E. coli, Clostridium spp, Eryspelothrix rhusiopathiae. Methods of preparing such formulations are described in, e.g., Remington's Pharmaceutical Sciences, Mack Publishing Company, Easton, Pa., 18 Edition, 1990.

The vaccines of the present invention may be prepared as injectables, either as liquid solutions or suspensions. Solid forms suitable for solution in or suspension in liquid vehicles prior to injection may also be prepared. The preparation may also be emulsified or the active ingredient encapsulated in liposome vehicles. Vaccines suitable for mucosal delivery, such as oral or nasal delivery, can also be readily formulated. The active immunogenic ingredient is generally mixed with a compatible pharmaceutical vehicle, such as, for example, water, saline, dextrose, glycerol, ethanol, or the like, and combinations thereof. In addition, if desired, the vehicle may contain minor amounts of auxiliary substances such as wetting or emulsifying agents and pH buffering agents.

Adjuvants which enhance the effectiveness of the vaccine may also be added to the formulation. Adjuvants may include for example, aluminum hydroxide, alum, muramyl dipeptides, avridine, Freund's adjuvant, incomplete Freund's adjuvant (ICFA), dimethyldioctadecyl ammonium bromide (DDA), oils, oil-in-water emulsions, saponins, cytokines, and other substances known in the art. Such adjuvants are well known and commercially available from a number of sources, e.g., Difco, Pfizer Animal Health, Newport Laboratories, etc.

Also useful herein is a triple adjuvant formulation as described in, e.g., U.S. Pat. No. 9,061,001, incorporated herein by reference in its entirety. The triple adjuvant formulation includes a host defense peptide, in combination with a polyanionic polymer such as a polyphosphazene, and a nucleic acid sequence possessing immunostimulatory properties (ISS), such as an oligodeoxynucleotide molecule with or without a CpG motif (a cytosine followed by guanosine and linked by a phosphate bond) or the synthetic dsRNA analog poly(I:C).

Examples of host defense peptides for use in the combination adjuvant, as well as individually with the antigen include, without limitation, HH2 (VQLRIRVAVIRA, SEQ ID NO:2); 1002 (VQRWLIVWRIRK, SEQ ID NO:3); 1018 (VRLIVAVRIWRR, SEQ ID NO:4); Indolicidin (ILPWKWPWWPWRR, SEQ ID NO:5); HH111 (ILKWKWPWWPWRR, SEQ ID NO:6); HH113 (ILPWKKPWWPWRR, SEQ ID NO:7); HH970 (ILKWKWPWWKWRR, SEQ ID NO:8); HH1010 (ILRWKWRWWRWRR, SEQ ID NO:9); Nisin Z (Ile-Dhb-Ala-Ile-Dha-Leu-Ala-Abu-Pro-Gly-Ala-Lys-Abu-Gly-Ala-Leu-Met-Gly-Ala-Asn-Met-Lys-Abu-Ala-Abu-Ala-Asn-Ala-Ser-Ile-Asn-Val-Dha-Lys, SEQ ID NO:10); JKI (VFLRRIRVIVIR; SEQ ID NO:11); JK2 (VFWRRIRVWVIR; SEQ ID NO:12); JK3 (VQLRAIRVRVIR; SEQ ID NO:13); JK4 (VQLRRIRVWVIR; SEQ ID NO:14); JK5 (VQWRAIRVRVIR; SEQ ID NO:15); and JK6 (VQWRRIRVWVIR; SEQ ID NO:16). Any of the above peptides, as well as fragments and analogs thereof, that display the appropriate biological activity, such as the ability to modulate an immune response, such as to enhance an immune response to a co-delivered antigen, will find use herein.

Exemplary, non-limiting examples of ISSs for use in the triple adjuvant composition, or individually include, CpG oligonucleotides or non-CpG molecules. By “CpG oligonucleotide” or “CpG ODN” is meant an immunostimulatory nucleic acid containing at least one cytosine-guanine dinucleotide sequence (i.e., a 5′ cytidine followed by 3′ guanosine and linked by a phosphate bond) and which activates the immune system. An “unmethylated CpG oligonucleotide” is a nucleic acid molecule which contains an unmethylated cytosine-guanine dinucleotide sequence (i.e., an unmethylated 5′ cytidine followed by 3′ guanosine and linked by a phosphate bond) and which activates the immune system. A “methylated CpG oligonucleotide” is a nucleic acid which contains a methylated cytosine-guanine dinucleotide sequence (i.e., a methylated 5′ cytidine followed by a 3′ guanosine and linked by a phosphate bond) and which activates the immune system. CpG oligonucleotides are well known in the art and described in, e.g., U.S. Pat. Nos. 6,194,388; 6,207,646; 6,214,806; 6,218,371; 6,239,116; and 6,339,068; PCT Publication No. WO 01/22990; PCT Publication No. WO 03/015711; US Publication No. 20030139364, which patents and publications are incorporated herein by reference in their entireties.

Examples of such CpG oligonucleotides include, without limitation, 5′TCCATGACGTTCCTGACGTT3′ (SEQ ID NO:17), termed CpG ODN 1826, a Class B CpG; 5′TCGTCGTTGTCGTTTTGTCGTT3′ (SEQ ID NO:18), termed CpG ODN 2007, a Class B CpG; 5′TCGTCGTTTTGTCGTTTTGTCGTT3′ (SEQ ID NO:19), also termed CPG 7909 or 10103, a Class B CpG; 5′ GGGGACGACGTCGTGGGGGGG 3′ (SEQ ID NO:20), termed CpG 8954, a Class A CpG; and 5′TCGTCGTTTTCGGCGCGCGCCG 3′ (SEQ ID NO:21), also termed CpG 2395 or CpG 10101, a Class C CpG. All of the foregoing class B and C molecules are fully phosphorothioated.

Non-CpG oligonucleotides for use in the present composition include the double stranded polyriboinosinic acid:polyribocytidylic acid, also termed poly(I:C); and a non-CpG oligonucleotide 5′AAAAAAGGTACCTAAATAGTATGTTTCTGAAA3′ (SEQ ID NO:22).

Polyanionic polymers for use in the triple combination adjuvants or alone include polyphosphazines. Typically, polyphosphazenes for use with the present adjuvant compositions will either take the form of a polymer in aqueous solution or a polymer microparticle, with or without encapsulated or adsorbed substances such as antigens or other adjuvants. For example, the polyphosphazene can be a soluble polyphosphazene, such as a polyphosphazene polyelectrolyte with ionized or ionizable pendant groups that contain, for example, carboxylic acid, sulfonic acid or hydroxyl moieties, and pendant groups that are susceptible to hydrolysis under conditions of use to impart biodegradable properties to the polymer. Such polyphosphazene polyelectrolytes are well known and described in, for example, U.S. Pat. Nos. 5,494,673; 5,562,909; 5,855,895; 6,015,563;and 6,261,573, incorporated herein by reference in their entireties. Alternatively, polyphosphazene polymers in the form of cross-linked microparticles will also find use herein. Such cross-linked polyphosphazene polymer microparticles are well known in the art and described in, e.g., U.S. Pat. Nos. 5,053,451; 5,149,543; 5,308,701; 5,494,682; 5,529,777; 5,807,757; 5,985,354; and 6,207,171, incorporated herein by reference in their entireties.

Examples of particular polyphosphazene polymers for use herein include poly[di(sodium carboxylatophenoxy)phosphazene] (PCPP) and poly(di-4-oxyphenylproprionate)phosphazene (PCEP), in various forms, such as the sodium salt, or acidic forms, as well as a polymer composed of varying percentages of PCPP or PCEP copolymer with hydroxyl groups, such as 90:10 PCPP/OH. Methods for synthesizing these compounds are known and described in the patents referenced above, as well as in Andrianov et al., Biomacromolecules (2004) 5:1999; Andrianov et al., Macromolecules (2004) 37:414; Mutwiri et al., Vaccine (2007) 25:1204.

Additional adjuvants include chitosan-based adjuvants, and any of the various saponins, oils, and other substances known in the art, such as AMPHIGEN™ which comprises de-oiled lecithin dissolved in an oil, usually light liquid paraffin. In vaccine preparations AMPHIGEN™ is dispersed in an aqueous solution or suspension of the immunizing antigen as an oil-in-water emulsion. Other adjuvants are LPS, bacterial cell wall extracts, bacterial DNA, synthetic oligonucleotides and combinations thereof (Schijns et al., Curr. Opi. Immunol. (2000) 12:456), Mycobacterial phlei (M. phlei) cell wall extract (MCWE) (U.S. Pat. No. 4,744,984), M. phlei DNA (M-DNA), M-DNA-M phlei cell wall complex (MCC). For example, compounds which may serve as emulsifiers herein include natural and synthetic emulsifying agents, as well as anionic, cationic and nonionic compounds. Among the synthetic compounds, anionic emulsifying agents include, for example, the potassium, sodium and ammonium salts of lauric and oleic acid, the calcium, magnesium and aluminum salts of fatty acids (i.e., metallic soaps), and organic sulfonates such as sodium lauryl sulfate. Synthetic cationic agents include, for example, cetyltrimethylammonium bromide, while synthetic nonionic agents are exemplified by glyceryl esters (e.g., glyceryl monostearate), polyoxyethylene glycol esters and ethers, and the sorbitan fatty acid esters (e.g., sorbitan monopalmitate) and their polyoxyethylene derivatives (e.g., polyoxyethylene sorbitan monopalmitate). Natural emulsifying agents include acacia, gelatin, lecithin and cholesterol.

Other suitable adjuvants can be formed with an oil component, such as a single oil, a mixture of oils, a water-in-oil emulsion, or an oil-in-water emulsion. The oil may be a mineral oil, a vegetable oil, or an animal oil. Mineral oil, or oil-in-water emulsions in which the oil component is mineral oil are preferred. Another oil component are the oil-in-water emulsions sold under the trade name of EMULSIGEN™, such as but not limited to EMULSIGEN PLUS™, comprising a light mineral oil as well as 0.05% formalin, and 30 ng/mL gentamicin as preservatives), available from MVP Laboratories, Ralston, Neb. Also of use herein is an adjuvant known as “VSA3” which is a modified form of EMULSIGEN PLUS™ which includes DDA (see, U.S. Pat. No. 5,951,988, incorporated herein by reference in its entirety). Suitable animal oils include, for example, cod liver oil, halibut oil, menhaden oil, orange roughy oil and shark liver oil, all of which are available commercially. Suitable vegetable oils, include, without limitation, canola oil, almond oil, cottonseed oil, corn oil, olive oil, peanut oil, safflower oil, sesame oil, soybean oil, and the like.

Alternatively, a number of aliphatic nitrogenous bases can be used as adjuvants with the vaccine formulations. For example, known immunologic adjuvants include amines, quaternary ammonium compounds, guanidines, benzamidines and thiouroniums (Gall, D. (1966) Immunology 11:369 386). Specific compounds include dimethyldioctadecylammonium bromide (DDA) (available from Kodak) and N,N-dioctadecyl-N,N-bis(2-hydroxyethyl)propanediamine (“AVRIDINE”). The use of DDA as an immunologic adjuvant has been described; see, e.g., the Kodak Laboratory Chemicals Bulletin 56(1):1 5 (1986); Adv. Drug Deliv. Rev. 5(3):163 187 (1990); J. Controlled Release 7:123 132 (1988); Clin. Exp. Immunol. 78(2):256 262 (1989); J. Immunol. Methods 97(2):159 164 (1987); Immunology 58(2):245 250 (1986); and Int. Arch. Allergy Appl. Immunol. 68(3):201 208 (1982). AVRIDINE is also a well-known adjuvant. See, e.g., U.S. Pat. No. 4,310,550, incorporated herein by reference in its entirety, which describes the use of N,N-higher alkyl-N′,N′-bis(2-hydroxyethyl)propane diamines in general, and AVRIDINE in particular, as vaccine adjuvants. U.S. Pat. No. 5,151,267 to Babiuk, incorporarted herein by reference in its entirety, and Babiuk et al. (1986) Virology 159:57 66, also relate to the use of AVRIDINE as a vaccine adjuvant.

PEDV immunogens may also be linked to a carrier in order to increase the immunogenicity thereof. Suitable carriers include large, slowly metabolized macro-molecules such as proteins, including serum albumins, keyhole limpet hemocyanin, immunoglobulin molecules, thyroglobulin, ovalbumin, and other proteins well known to those skilled in the art; polysaccharides, such as sepharose, agarose, cellulose, cellulose beads and the like; polymeric amino acids such as polyglutamic acid, polylysine, and the like; amino acid copolymers; and inactive virus particles.

PEDV immunogens may be used in their native form or their functional group content may be modified by, for example, succinylation of lysine residues or reaction with Cys-thiolactone. A sulfhydryl group may also be incorporated into the carrier (or antigen) by, for example, reaction of amino functions with 2-iminothiolane or the N-hydroxysuccinimide ester of 3-(4-dithiopyridyl propionate. Suitable carriers may also be modified to incorporate spacer arms (such as hexamethylene diamine or other bifunctional molecules of similar size) for attachment of peptides.

Furthermore, the PEDV immunogens may be formulated into vaccine compositions in either neutral or salt forms. Pharmaceutically acceptable salts include the acid addition salts (formed with the free amino groups of the active polypeptides) and which are formed with inorganic acids such as, for example, hydrochloric or phosphoric acids, or such organic acids as acetic, oxalic, tartaric, mandelic, and the like. Salts formed from free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Vaccine formulations will contain a “therapeutically effective amount” of the active ingredient, that is, an amount capable of eliciting an immune response in a subject to which the composition is administered. In the treatment and prevention of PEDV infection, a “therapeutically effective amount” is readily determined by one skilled in the art using standard tests. The PEDV immunogens will typically range from about 1% to about 95% (w/w) of the composition, or even higher or lower if appropriate. With the present vaccine formulations, .1 to 500 mg of active ingredient per ml, preferably 1 to 100 mg/ml, more preferably 10 to 50 mg/ml, such as 20 . . . 25 . . . 30 . . . 35 . . . 40, etc., or any number within these stated ranges, of injected solution should be adequate to raise an immunological response when a dose of 0.25 to 3 ml per animal is administered.

If an inactivated or attenuated preparation is used, the compositions will generally include 10² to 10¹² pfu, more particularly from 10⁴ to 10⁸ pfu, and preferably from 10⁵ to 10⁷ pfu of PEDV, or any pfu value within these stated ranges.

Preferably the dosage regime leads to antibodies with a neutralizing characteristic. An in vitro neutralization assay may be used to test for neutralizing antibodies (see, for example, Makadiya et al., Virology Journal (2016) 13:57 for an assay to test for PEDV neutralizing antibodies).

To immunize a subject, the vaccine is generally administered parenterally, usually by intramuscular injection. Other modes of administration, however, such as subcutaneous, intraperitoneal and intravenous injection, are also acceptable. The quantity to be administered depends on the animal to be treated, the capacity of the animal's immune system to synthesize antibodies, and the degree of protection desired. Effective dosages can be readily established by one of ordinary skill in the art through routine trials establishing dose response curves. The subject is immunized by administration of the vaccine in at least one dose, and preferably two or more doses. Moreover, the animal may be administered as many doses as is required to maintain a state of immunity to infection.

In one embodiment, the vaccines are administered to pregnant sows prior to farrowing in order to confer passive immunity to piglets born to the sows. If so, typically the vaccines will be administered anytime within 8 weeks of farrowing, such as beginning at 8, 7, 6, 5, 4, 3, 2, 1, 0.5 weeks before farrowing, such as between 1-6 weeks prior to farrowing, such as at 4-6 weeks prior to farrowing, optionally with at least one additional dose at 1, 2, 3 weeks, etc. before giving birth.

Additional vaccine formulations which are suitable for other modes of administration include suppositories and, in some cases, aerosol, intranasal, oral formulations, and sustained release formulations. For suppositories, the vehicle composition will include traditional binders and carriers, such as, polyalkaline glycols, or triglycerides. Such suppositories may be formed from mixtures containing the active ingredient in the range of about 0.5% to about 10% (w/w), preferably about 1% to about 2%. Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Controlled or sustained release formulations are made by incorporating the protein into carriers or vehicles such as liposomes, nonresorbable impermeable polymers such as ethylenevinyl acetate copolymers and Hytrel copolymers, swellable polymers such as hydrogels, or resorbable polymers such as collagen and certain polyacids or polyesters such as those used to make resorbable sutures. The PEDV immunogens can also be delivered using implanted mini-pumps, well known in the art.

An alternative route of administration involves gene therapy or nucleic acid immunization. Thus, nucleotide sequences (and accompanying regulatory elements) encoding the subject PEDV immunogens can be administered directly to a subject for in vivo translation thereof. Alternatively, gene transfer can be accomplished by transfecting the subject's cells or tissues ex vivo and reintroducing the transformed material into the host. Nucleic acid can be directly introduced into the host organism, i.e., by injection (see International Publication No. WO/90/11092; and Wolff et al. (1990) Science 247:1465-1468). Liposome-mediated gene transfer can also be accomplished using known methods. See, e.g., Hazinski et al. (1991) Am. J. Respir. Cell Mol. Biol. 4:206-209; Brigham et al. (1989) Am. J. Med. Sci. 298:278-281; Canonico et al. (1991) Clin. Res. 39:219A; and Nabel et al. (1990) Science 1990) 249:1285-1288. Targeting agents, such as antibodies directed against surface antigens expressed on specific cell types, can be covalently conjugated to the liposomal surface so that the nucleic acid can be delivered to specific tissues and cells susceptible to infection.

Diagnostic Assays

Antibodies and immunogens, produced as described above, can be used in vivo, i.e., injected into subjects suspected of having PEDV disease, for diagnostic or therapeutic uses. The use of antibodies for in vivo diagnosis is well known in the art. The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used. Localization of the label within the patient allows determination of the presence of the disease.

The antibodies can also be used in standard in vitro immunoassays, to screen biological samples such as blood and/or tissues for the presence or absence of PEDV infection. Thus, the antibodies produced as described above, can be used in assays to diagnose PEDV disease. The antibodies can be used as either the capture component and/or the detection component in the assays, as described further below. Thus, the presence of PEDV disease can be determined by the presence of PEDV antigens and/or anti-PEDV antibodies.

For example, the presence of PEDV antigens antigens can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as enzyme-linked immunosorbent assays (“ELISAs”); biotin/avidin type assays; radioimmunoassays; immunoelectrophoresis; immunoprecipitation, etc. The reactions generally include revealing labels such as fluorescent, chemiluminescent, radioactive, or enzymatic labels or dye molecules, or other methods for detecting the formation of a complex between the antigens and the antibodies described above.

Assays can also be conducted in solution, such that the antigens and antibodies thereto form complexes under precipitating conditions. The precipitated complexes can then be separated from the test sample, for example, by centrifugation. The reaction mixture can be analyzed to determine the presence or absence of antibody-antigen complexes using any of a number of standard methods, such as those immunodiagnostic methods described above.

Kits

The invention also provides kits comprising one or more containers of compositions of the invention. Compositions can be in liquid form or can be lyophilized, as can individual antigens. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may further include a third component comprising an adjuvant.

The kit can also comprise a package insert containing written instructions for methods of inducing immunity or for treating infections. The package insert can be an unapproved draft package insert or can be a package insert approved by the Food and Drug Administration (FDA) or other regulatory body.

The invention also provides a delivery device pre-filled with the immunogenic compositions of the invention.

Similarly, antibodies can be provided in kits, with suitable instructions and other necessary reagents, in order to conduct immunoassays as described above. The kit can also contain, depending on the particular immunoassay used, suitable labels and other packaged reagents and materials (i.e. wash buffers and the like). Standard immunoassays, such as those described above, can be conducted using these kits.

### 3. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Materials and Methods

### PEDV and Cell Culture:

Vero 76 cells were obtained from the American Type Culture Collection (ATCC). The cells were grown in complete DMEM and passaged twice a week using Versene-trypsin. Confluent monolayers were infected with PEDV CO 025 isolate, obtained from the USDA. After infection, supernatants and cell pellets were separated by centrifuge at 2000×g for 15 minutes at 10° C. The cell pellet was resuspended in a small volume of supernatant. The cell pellet and supernatant were stored at −80° C.

For preparation of viral challenge the cell pellets were diluted in DMEM and the viral titer determined by TCID₅₀. The challenge material was diluted to a titer of 3×10² pfu/ml and 1 ml was given orally per piglet.

For preparation of the vaccine, the supernatants were thawed at 37° C. in a water bath and subsequently centrifuged at 2671 g for 20 minutes at 4° C. to remove cellular debris. Virus was concentrated from the supernatants by centrifugation at 28,000 rpm for 2 hours in a Beckman Ultracentrifuge. After centrifugation the remaining supernatants were discarded and viral pellets resuspended in 75 μl phosphate-buffered saline (4° C. overnight), and stored at minus 80° C. Virus titers were confirmed by PCR.

For vaccines, PEDV from the viral pellets was inactivated using beta-propiolactone (BPL) at a final concentration of 0.1% (4° C. overnight). Inactivation was verified by in vitro cell culture using three passages, as well as in neonatal pigs exposed to 10× the dose of the experimental vaccine. Bradford assays were used to determine the protein concentration.

### PEDV Challenge Model:

Healthy, pregnant sows of different parities were obtained from Prairie Swine Centre and housed in farrowing crates and had previously been vaccinated for parvovirus, PCV and E. coli. Sows were randomly assigned to groups and housed in farrowing crates at a maximum of four sows per room. Sows were fed a commercial sow diet with access to water ad libitum. Sows were induced with oxytocin to synchronize farrowing. Neonatal piglets received iron injections and had access to water ad libitum. Litter sizes were limited to 12 piglets per sow using cross-fostering or humane euthanasia of the runts.

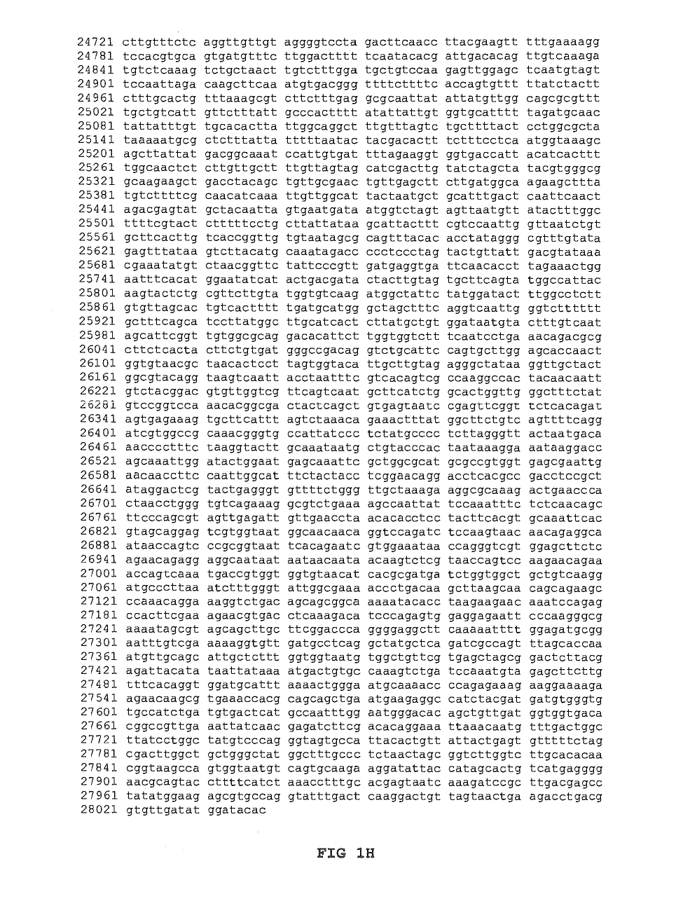

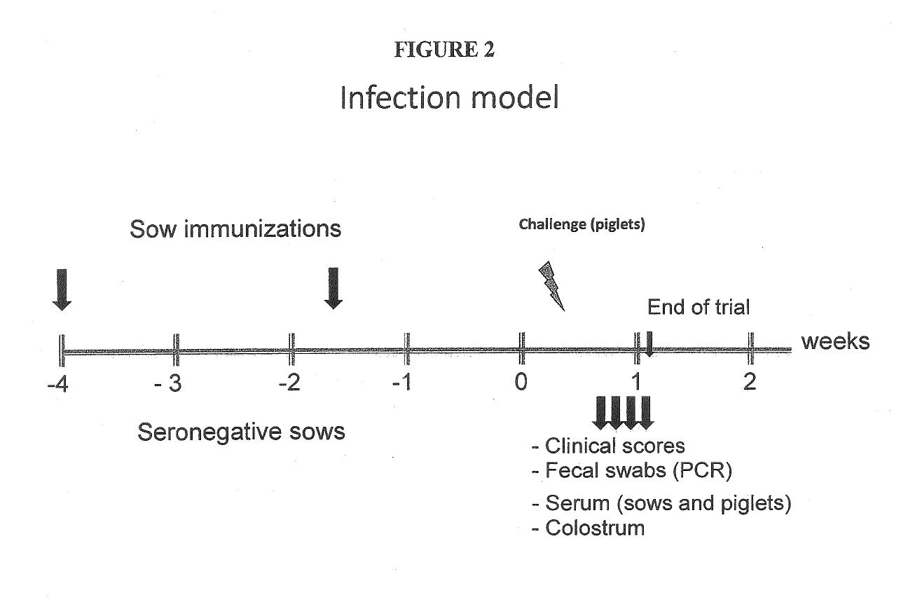

Pigs were infected orally either on day 1 or day 5 of life (See, FIG. 2). All studies were performed in a double-blinded fashion. Animals were monitored twice daily for clinical symptoms and, if needed, humanely euthanized after reaching endpoints as defined in the Animal Ethics Protocol. Fecal swabs were collected daily to determine viral shedding in feces. Colostrum and serum samples were collected prior to challenge and at the end of the trial to determine antibody titers. All animals were weighed daily to determine daily weight gain or loss. Within 24 hours of infection, neonatal piglets displayed apathy, diarrhea and significant weight loss. Moribund or dead pigs could be found starting at day 1 post infection all the way to 9 days post infection. Post mortem examination revealed extended, oedematous small and large intestines filled with milky-white gut content. When infected at day 1 of life, mortality reached up to 100%. When infected between 4-5 days of age, overall mortality ranged between 50-60%. Surviving piglets showed reduced weight gain for the next 8-10 days with a poor overall body condition. Viral shedding was detected in feces of infected piglets for up to 6 days post infection.

### Serum Anti-PEDV S1 Antibody Detection:

Serum anti-PEDV IgG titers were measured in piglets on the day of PEDV challenge by ELISA using PEDV SI purified recombinant protein as antigen, produced in human embryo kidney (HEK) 293 cells as described in Makadiya et al., Virology Journal (2016) 13:57. Sow serum IgG titers were measured at the time of farrowing.

### Vaccine Formulation:

Vaccines were formulated with a commercially available alum (aluminum hydroxide and magnesium hydroxide; Pierce Imject™ Alum adjuvant, Fischer Scientific), or a combination adjuvant (triple combination adjuvant) including poly I:C, the polyphosphazene PCEP, and the host defense peptide 1002 (VQRWLIVWRIRK, SEQ ID NO:3) (see, U.S. Pat. No. 9,061,001, incorporated herein by reference in its entirety). Vaccines were formulated prior to injection by mixing adjuvant and inactivated virus at room temperature. Alum was used undiluted and was mixed 1:1 with the inactivated virus formulation. After 30 minutes, vaccine formulations were stored on ice until administered to the sows as described below. Alum is known to enhance a Th2-type immune response, which promotes humoral (antibody-mediated immunity), while the triple combination adjuvant promotes a balanced/Th-1 type of immunity, which facilitates cell-mediated and humoral immunity.

### Vaccination:

Individually ear-tagged sows were immunized with two doses of the experimental vaccine, a high dose (8×10⁵pfu/sow for vaccination trial I and 5×10⁵ for subsequent trials) and a low dose (8×10⁴pfu/sow). The vaccine was administered intramuscularly in the neck region in a total volume of 4 ml, half of the vaccine (2 ml) per side. The vaccine was administered in a two-week interval, at four and two weeks prior to farrowing. All immunized sows were monitored daily for adverse reactions to the vaccine. Farrowing was induced with Planate™, a synthetic prostaglandin analogue for use in swine. Oxytocin was used when needed.

### Example 1

### Vaccination Trials I and II

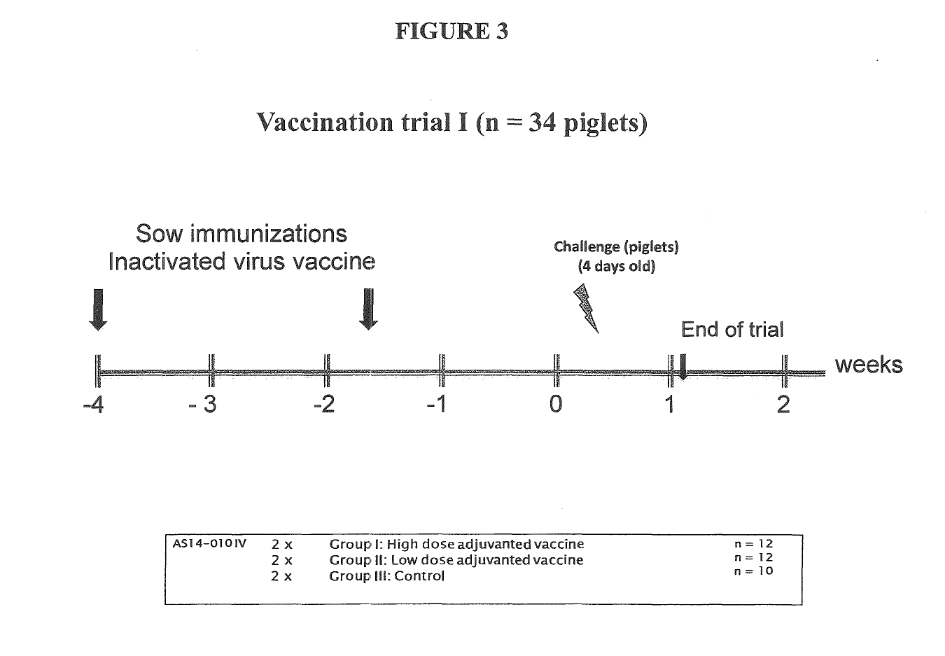

For vaccination trial I, a total of three sows were immunized as follows (see, FIG. 3): In Group I (consisting of one sow; 12 piglets), one sow was immunized with a high dose of the inactivated vaccine adjuvanted with the triple combination adjuvant; In Group II (consisting of one sow; 12 piglets), one sow was immunized with a low dose of the inactivated vaccine adjuvanted with the triple combination adjuvant; In Group III (consisting of one sow; 10 piglets), one sow was used as a control.

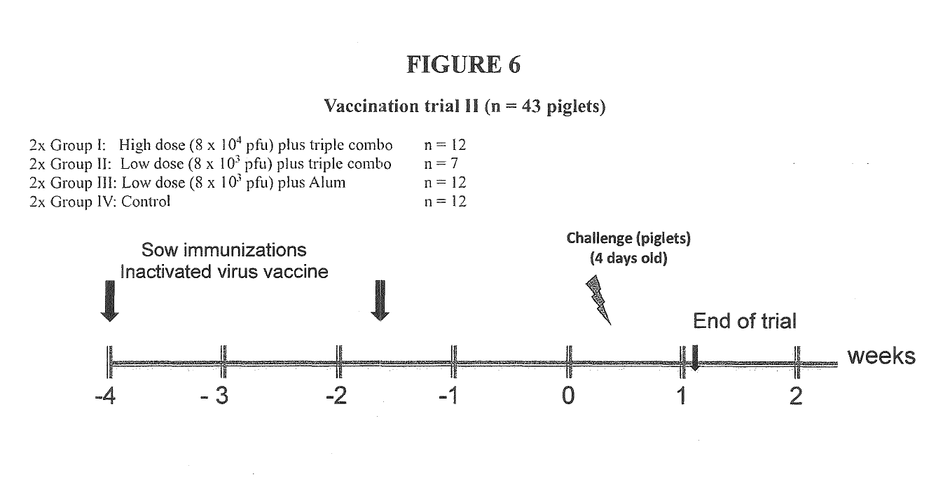

For vaccination trial II, a total of four sows were immunized as follows (see, FIG. 6): In Group I (consisting of one sow; 12 piglets), one sow was immunized with a high dose of the inactivated vaccine adjuvanted using the triple combination adjuvant; In Group II (consisting of one sow; seven piglets), one sow was immunized with a low dose of the inactivated vaccine adjuvanted using the triple combination adjuvant; In Group III (consisting of one sow; 12 piglets), one sow was immunized with a low dose of the inactivated vaccine adjuvanted using alum; In Group IV (consisting of one sow; 12 piglets), one sow served as a control.

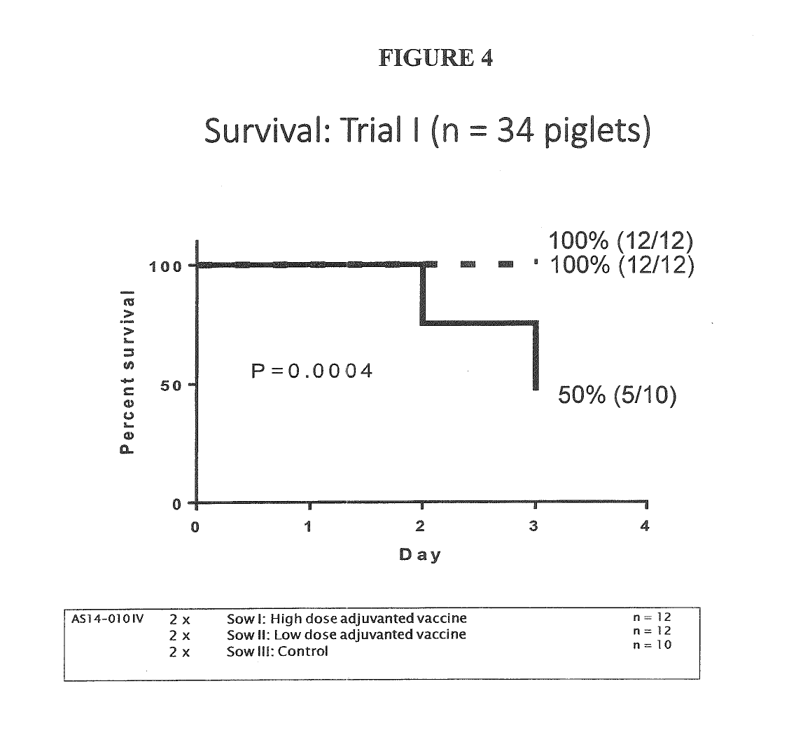

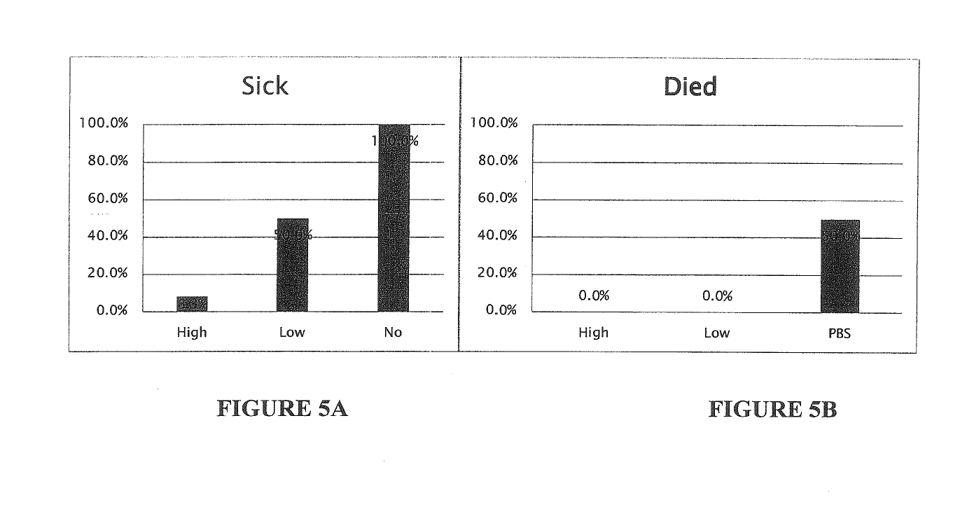

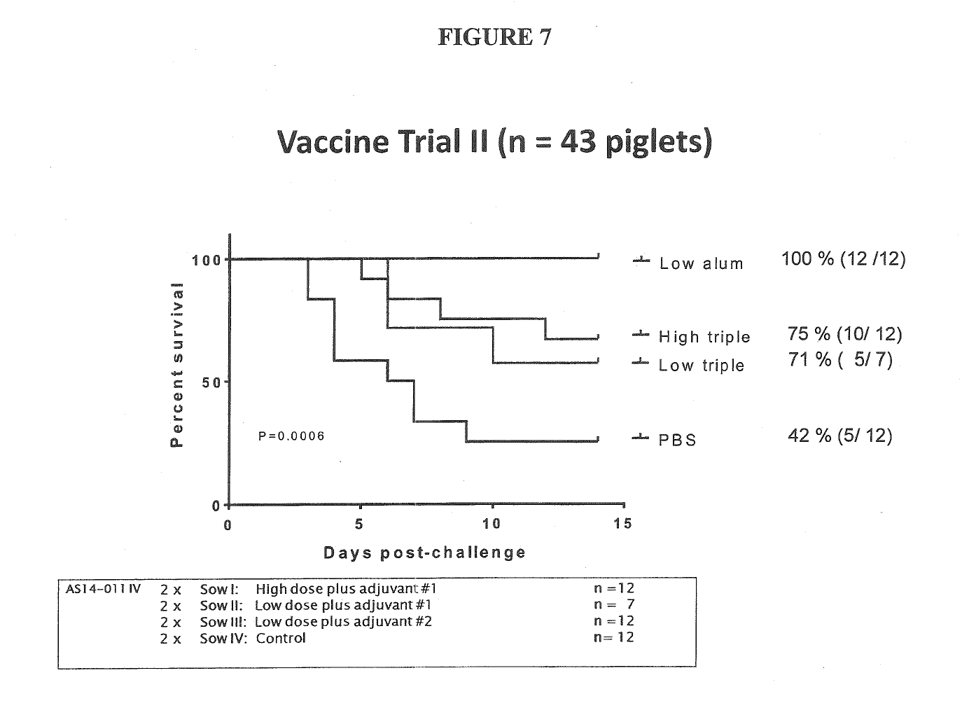

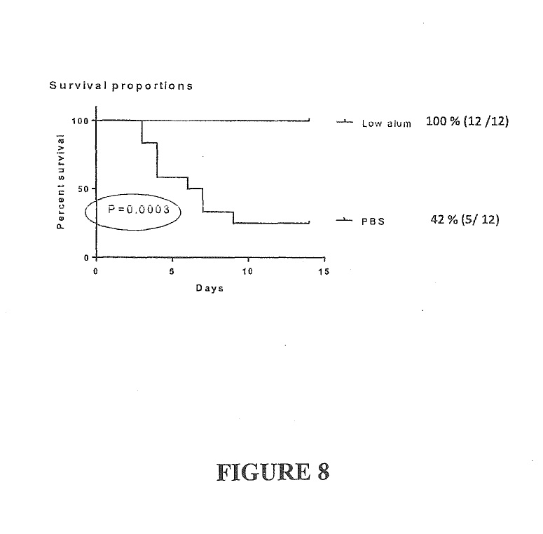

As can be seen from the data in vaccine trial I, all piglets from the sows vaccinated with the vaccine formulated with the triple adjuvant survived while 50% of the piglets from the control sows died (see, FIGS. 4 and 5A and 5B). In vaccine trial II, all piglets from the sow vaccinated with the vaccine formulated with alum survived the infection, while approximately 42% of the piglets from the control sow died (see, FIGS. 7 and 8).

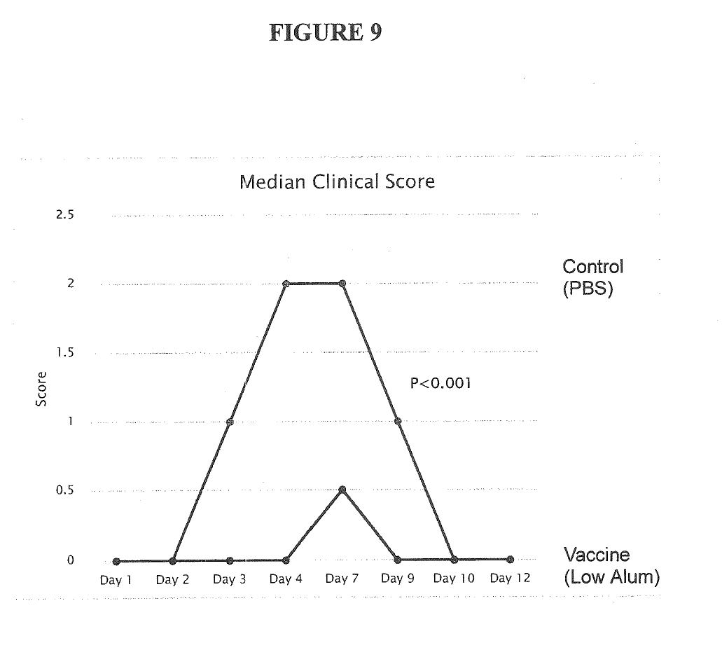

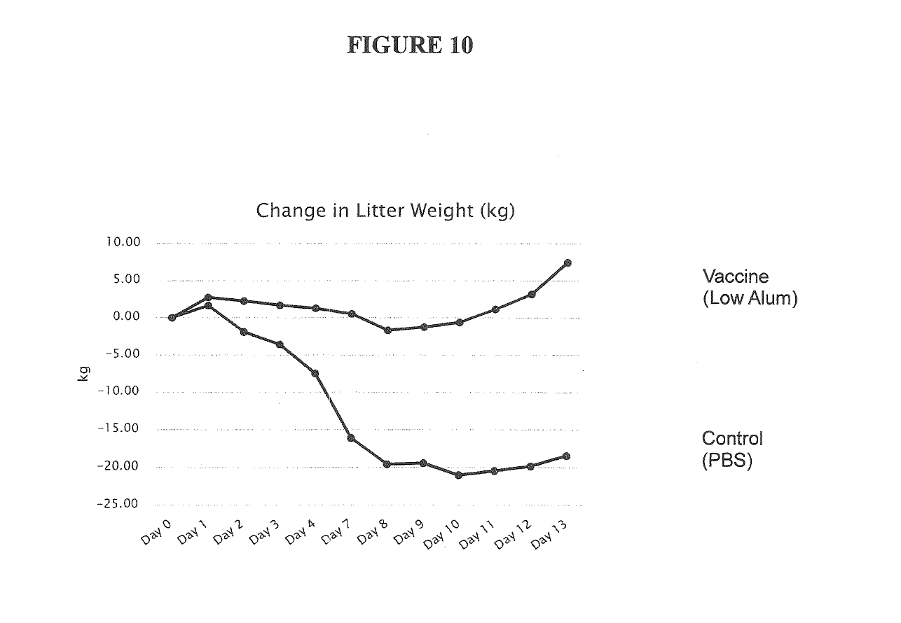

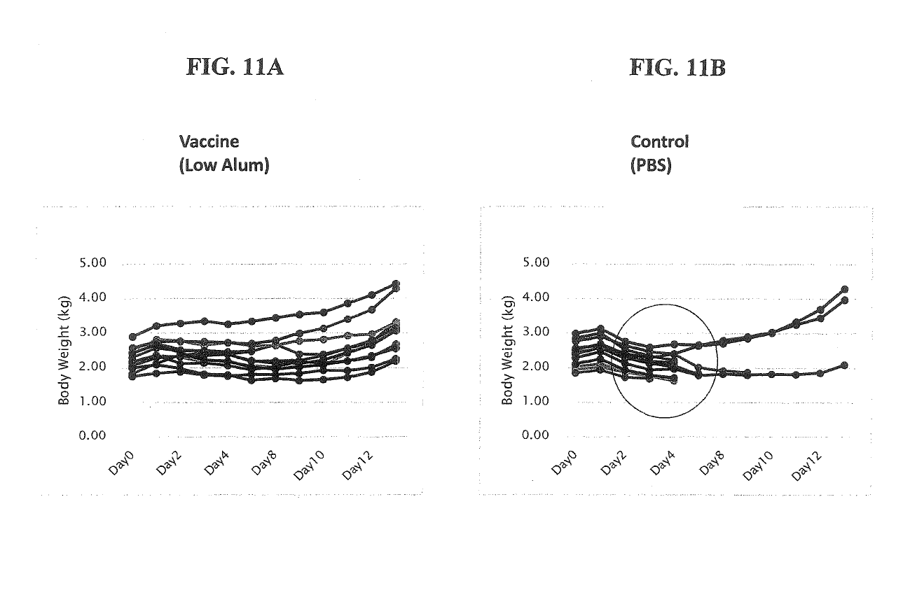

Additionally, in vaccine trial II, vaccination of sows with the vaccine formulated with the triple combination adjuvant resulted in about 70-75% survival of the piglets, depending on the dose (FIG. 7). Piglets from the sow vaccinated with the inactivated vaccine formulated with alum showed fewer clinical symptoms than piglets from the control sow with a short-lasting decrease in weight gain, compared to the control piglets which clearly showed significant losses in weight gain with many of the piglets plateauing at a low overall body weight. (FIGS. 9, 10 and 11).

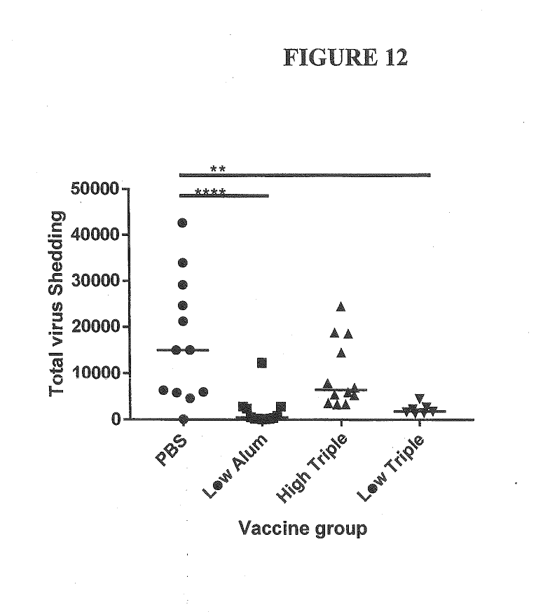

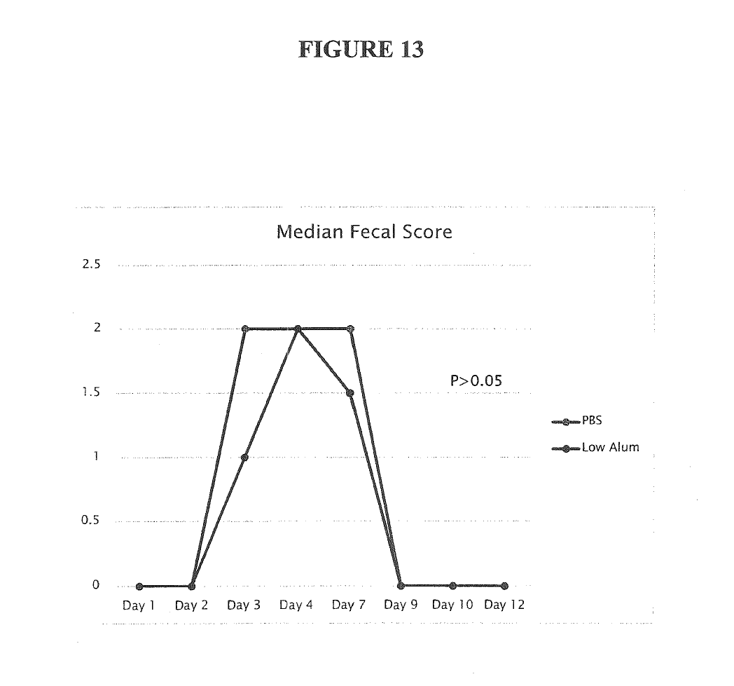

To assess virus shedding in piglets, the level of viral genome or N-gene transcripts in feces of piglets collected at Days 1 and 4 post-challenge, was determined by a PEDV N gene-based real-time RT-PCR. The cycle thresholds (CT) values were converted to the number of viral particles present using a standard curve. Viral shedding for each piglet was summed for days 1 to 4 post-challenge. If the piglet died, the median level of shedding was used for the subsequence days. While all piglets displayed diarrhea for a period of time, piglets from vaccinated sows shed significantly less virus compared to piglets from control sows (FIG. 12). Very little or no virus was detected in fecal swabs from piglets of vaccinated sows between 1-4 days post-infection (FIG. 13).

Thus, in summary, the inactivated vaccine, when formulated with alum as an adjuvant, protected 100% of the progeny of the vaccinated sow against infection with PEDV. When formulated with the triple combination adjuvant, approximately 70-75% of the piglets from the vaccinated sows were protected.

### Example 2

### Vaccination Trial III

For vaccination trial III, a total of seven sows were immunized as described in the methods above. In Group I (consisting of four sows; 45 piglets) four sows were immunized with the low dose of the inactivated vaccine formulated in the alum adjuvant; In Group II (consisting of three sows; 35 piglets), three sows were treated with saline as a control.

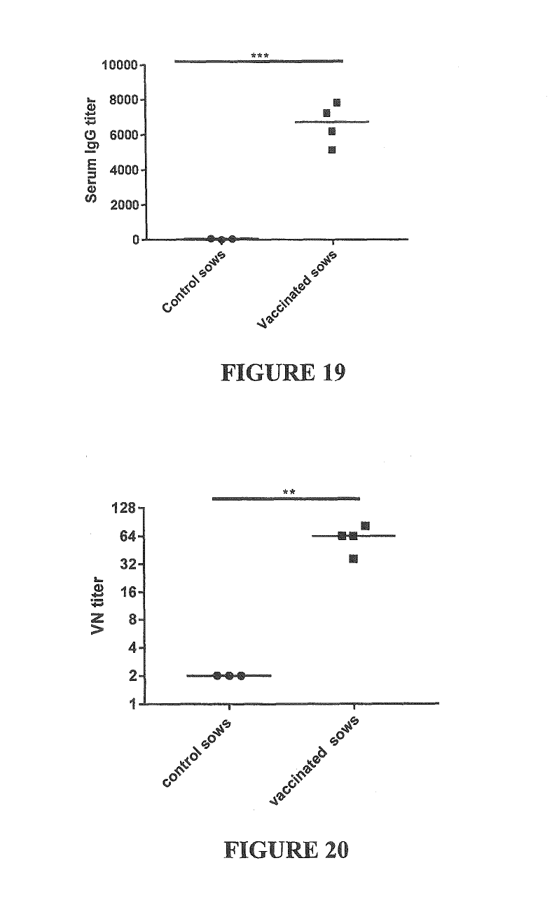

Serum IgG titers were measured in sows at the time of farrowing by ELISA using the recombinant purified S1 protein as an antigen. Results are shown in FIG. 19. A statistically significant difference was observed between the control and vaccinated groups of sows (p=0.0002).

Viral neutralization titers were measured in sows after farrowing. Results are shown in FIG. 20. A statistically significant difference was observed between the control and vaccinated groups of sows (p=0.0032).

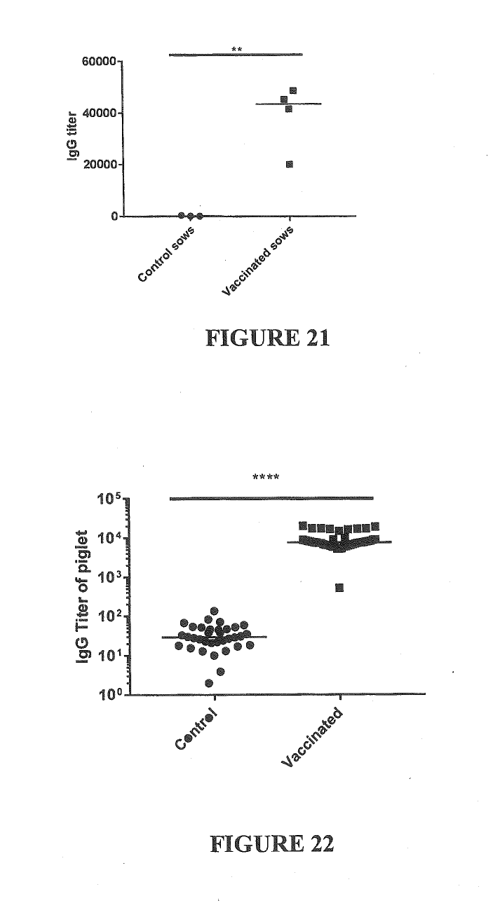

Sow colostrum IgG titers were measured after farrowing by ELISA using the recombinant S1 protein as an antigen. Results are shown in FIG. 21. A statistically significant difference was observed between the control and vaccinated groups of sows (p=0.0039).

Piglet serum IgG titers were measured on the day of PEDV challenge by ELISA using the PEDV S1 purified recombinant protein. As shown in FIG. 22, the IgG titers of piglets born to the four vaccinated sows (45 piglets) were higher than those from the three control sows (34 piglets).

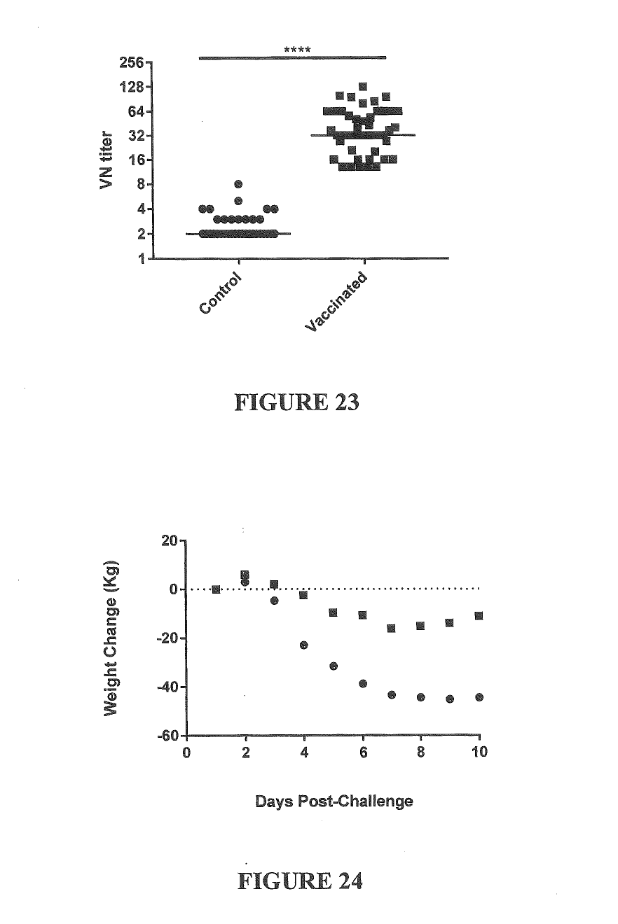

Viral neutralization titers of piglets from control and vaccinated sows were determined when piglets were five days old. Results are shown in FIG. 23. The median value between piglets from control and piglets from vaccinated sows was statistically significant (p<0.0001).

The weight change for the piglets from the vaccinated sows and control sows was also determined. This was done by summing the weights of the piglets from each group that were alive on each day and subtracting it from the initial litter weight. Results are shown in FIG. 24. As can be seen in FIG. 24, the piglets from control sows showed higher losses in weight gain than in piglets from vaccinated sows.

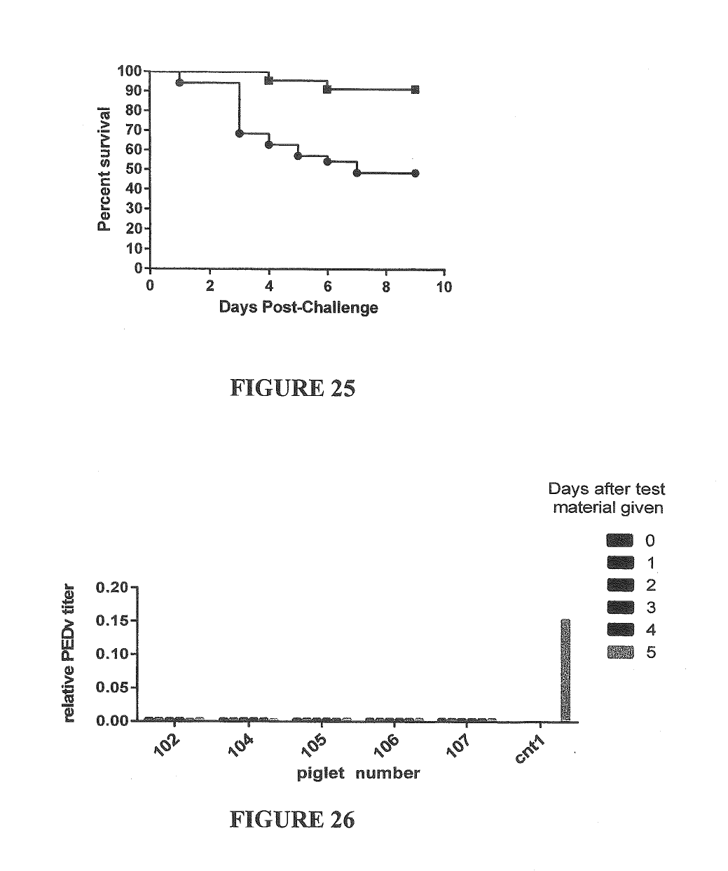

Survival curves of piglets from vaccinated and control sows were determined. As shown in FIG. 25, 91% of the piglets born to vaccinated sows survived, while 49% of the piglets born to control sows survived. The survival curves from the two groups were significantly different (p<0.0001).

Thus, the data evidences that neutralizing antibodies are involved in providing protection against PEDV, especially in neonatal pigs

### Example 3

### Vaccine Safety and Immunogenicity

In order to test whether the vaccine was effectively inactivated using beta-propiolactone, the following study was done. PEDV from the viral pellets was inactivated using beta-propiolactone at final concentrations of 0.01%, 0.05% and 0.1% and incubated for two, eight and 18 hours at 4° C. Inactivation was verified by in vitro cell culture using three passages. All concentrations and timepoints displayed inactivation of virus.

Inactivation was also confirmed using an in vivo virus detection assay in neonatal pigs exposed to 10× the dose of the inactivated vaccine. Five neonatal piglets, approximately 12 hours of age, were orally administered the vaccine. Piglets at this age are the most susceptible to the infection. The positive control was a piglet that had been infected with PEDV and sampled four days after challenge. As shown in FIG. 26, only the control piglet showed significant viral titers.

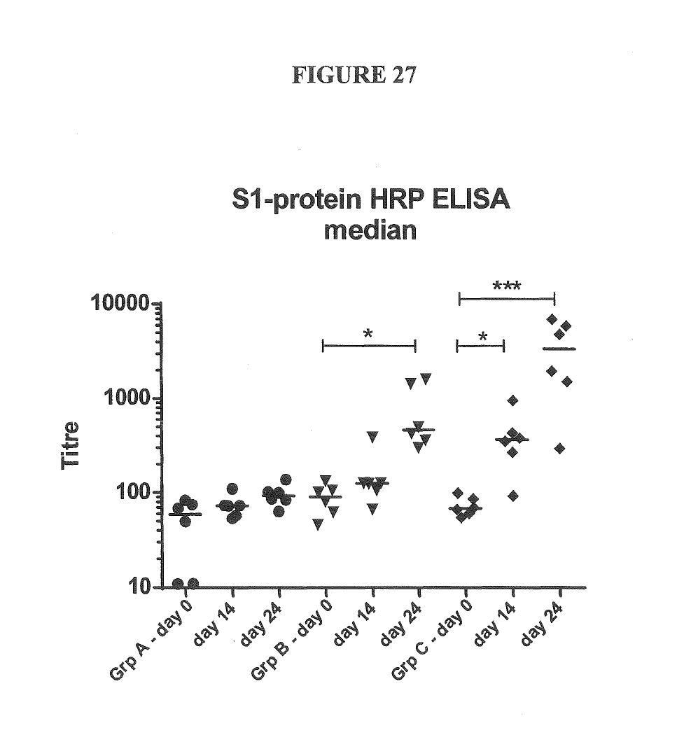

Additionally, since inactivation can lead to degradation of the vaccine virus and thus reduce the immunogenicity of the vaccine, an experiment was performed to assess the immunogenicity of vaccine doses in vivo. In particular, the immunogenicity of the vaccine after inactivation was tested by measuring serum anti-PEDV IgG titers in an ELISA using PEDV S1 recombinant, purified protein as antigen. Three groups of six piglets were administered the inactivated vaccine adjuvanted with alum. Group A was administered saline as a control. Group B was administered 2 ml of a dose of 5×10⁴ TCID 50/ml. Group C was administered 2 ml of a dose of 2×10⁵ TCID 50/ml. As can be seen in FIG. 27, both doses displayed significant immunogenicity as compared to the control.

Based on the above, the BPL inactivated PEDV vaccine is indeed safe and effective.

### Example 4

### Field Trial of PEDV Vaccine

A larger trial of the PEDV vaccine adjuvanted with alum is being conducted at three sites in Saskatchewan, Canada. Each site included vaccine groups and a control group. Sows were randomly assigned to groups and personnel conducting the field trials were blinded to the treatment groups.

In Phase I of the trials, safety of the vaccine was confirmed using 72 sows (24 sows per site). Three groups per site were used. Group I was given 5 × the vaccine does; Group II was given 1 × the vaccine does; and Group III was given adjuvant alone, administered as described above. The injection side was monitored at days 1, 2, 3 and 7 post injection. Reproductive safety was determined by monitoring the number of piglets; number born live; still births; and piglet health. Antibody titer in piglets is being determined every 3-6 months.

Phase II of the trials is being conducted to determine immunogenicity and efficacy. This study includes 524 sows at three different sites in Saskatchewan, Canada. Two groups of sows are being tested. Group I was given 1× the vaccine dose; Group II was given adjuvant alone.

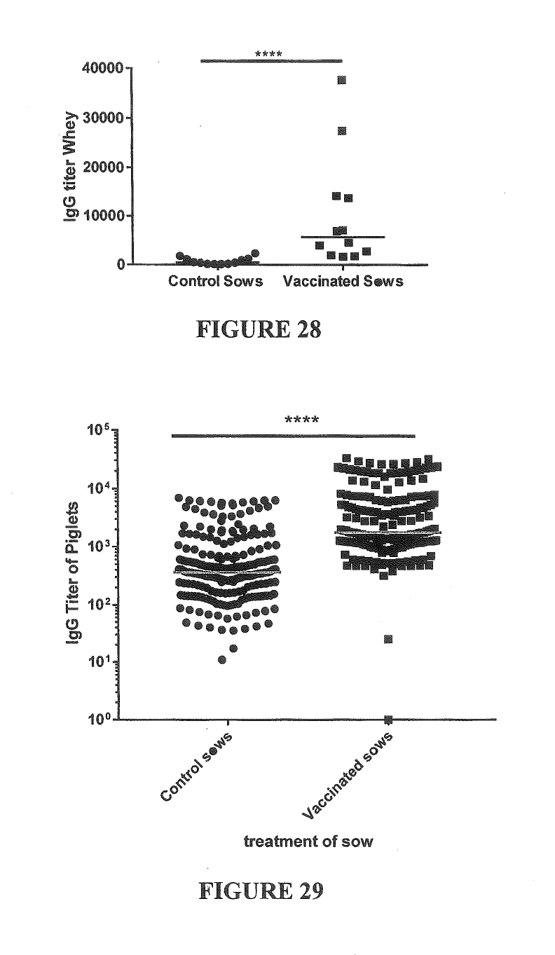

In one experiment, sow colostrum IgG titers were measured in whey from the two groups of sows, 12 sows/group, after farrowing by ELISA using the purified, recombinant SI protein as antigen, as described above. Results are shown in FIG. 28. A statistically significant difference was observed between the groups of sows (p<0.0001).

Additionally, piglet serum IgG titers were measured on the day of PEDV challenge by. ELISA using the purified, recombinant S1 protein as antigen. Data derived from nine challenge trials using a total of 18 vaccinated sows (207 piglets) and 18 control sows (209 piglets) are shown in FIG. 29. The median IgG titer from piglets of vaccinated sows was significantly different than that of piglets from control sows (P<0.0001).

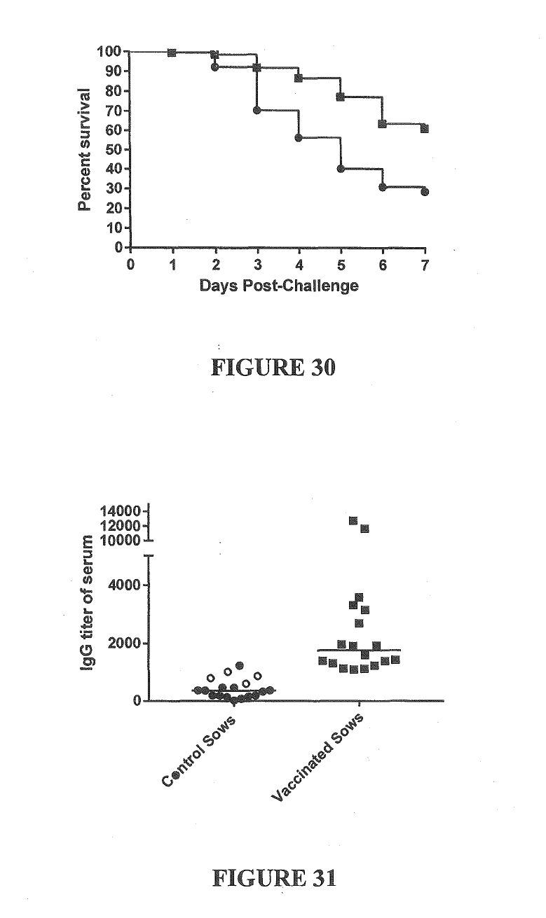

Survival of piglets from control sows and piglets from vaccinated sows was determined. Data derived from nine challenge trials using a total of 18 vaccinated sows (207 piglets) and 18 control sows (209 piglets) are shown in FIG. 30. The survival curve for piglets from vaccinated sows was significantly different than that of piglets from control sows (P<0.0001).

Sow serum IgG titers were measured at the time of farrowing, prior to challenge, by ELISA using the purified, recombinant SI protein as antigen. Results are shown in FIG. 31. A statistically significant difference was observed between the two groups of sows (P<0.0001).

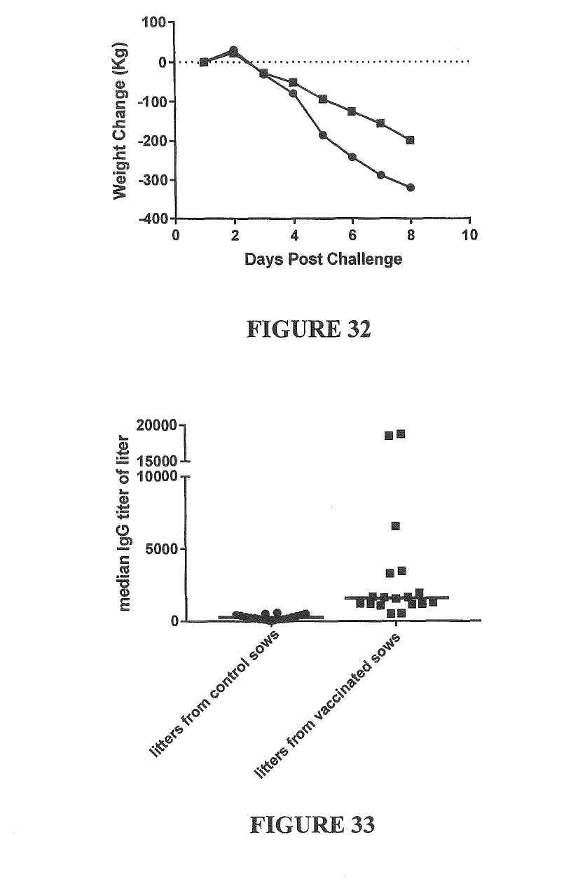

The weight changes of piglets from control sows and vaccinated sows were summed on each day after challenge and subtracted from the sum of the weight on the day of challenge (day 0). As shown in FIG. 32, the piglets from control sows showed higher losses in weight gain than in piglets from vaccinated sows.

Serum IgG titers in piglets prior to challenge was determined. Data derived from nine challenge trials using a total of 18 vaccinated sows (207 piglets) and 18 control sows (209 piglets) are shown in FIG. 33. A statistically significantly difference was observed between the litters from control and vaccinated sows (p<0.0001).

### Example 5

### Preparation of PEDV Cell Pellet Vaccine and Comparison to the Supernatant Vaccine

In order to determine whether a PEDV cell pellet vaccine was efficacious and comparable to the supernatant PEDV vaccine, the following experiment was conducted. PEDV was grown under standard conditions as described above and the cells and supernatant were collected. A slow speed centrifugation (2000×g for 15 minutes at 10° C.) was done to pellet cells and debris. The supernatant was removed and used to produce the standard vaccine as described above. The cell pellets from each flask were suspended in the residual culture supernatant and pellets from approximately 50 flasks were pooled (approximately 20 ml for 50 flasks). The cell pellet was stored at −80° C. until used.

To prepare the vaccine, the cell pellets were freeze/thawed and sonicated until material no longer settled in the tube. The concentration of cells was determined by TCID₅₀ and PCR.

The virus was inactivated using using beta-propiolactone (BPL) 0.1% for 18 hours as described above. Inactivation of the virus was confirmed in vitro by serial passage in Vero 76 cells three times.

To test the cell pellet vaccine four groups of sows, two sows per group were used as shown in Table 1.

| | Primary Vaccination | Boost vaccination |
|---|---|---|
| Group | (4 weeks before farrowing) | (2 weeks before farrowing) |
| A | alum only | alum only |
| B* | supernatant vaccine | supernatant vaccine |
| C* | cell pellet vaccine | cell pellet vaccine |
| D** | | supernatant vaccine |

*The supernatant and cell pellet vaccine contained 2 × 10⁵ viral particles formulated with alum.
**Sows in group D received a single vaccination with 1 × 10⁶ viral particles formulated with alum.

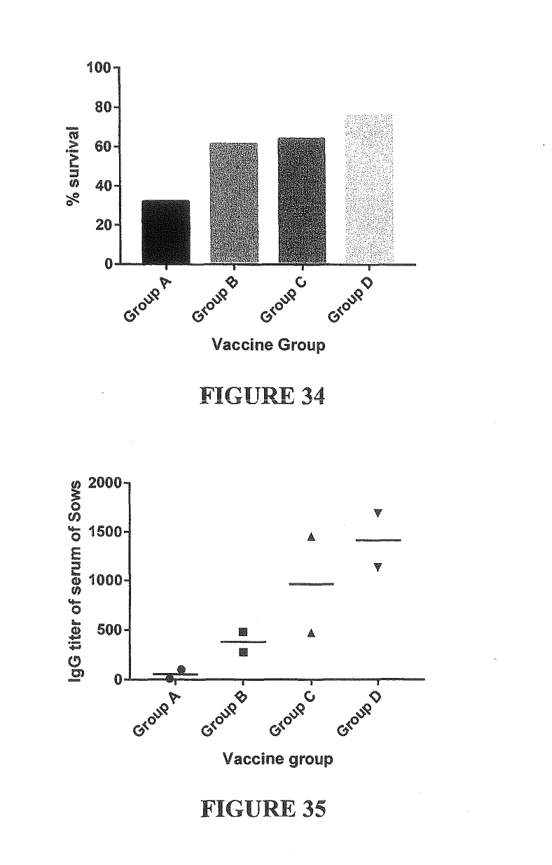

FIG. 34 shows the percentage survival of piglets at seven days of age in the various groups. As can be seen, survival of piglets from both Groups B and C were comparable and were higher than those administered the control vaccine. A higher percentage of piglets from Group D, given the higher dose of vaccine, survived.

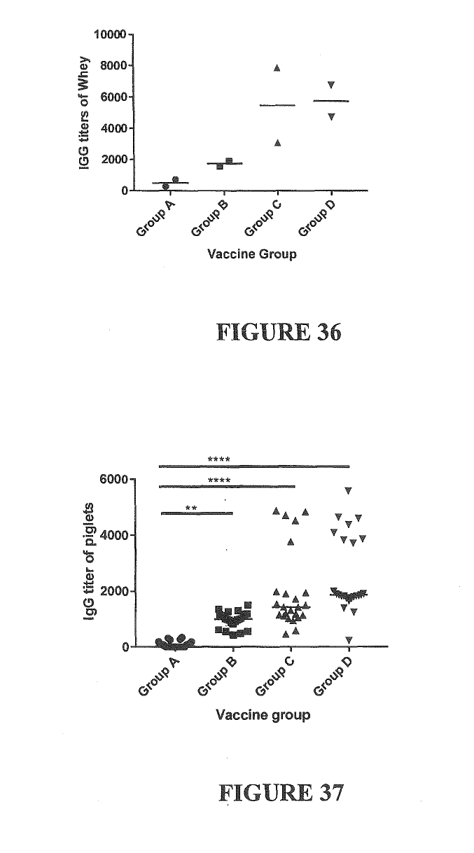

FIGS. 35 and 36 show the serum IgG titers from sows (FIG. 35), and IgG titers from whey from sows (FIG. 36) of the various groups. FIG. 37 shows the serum IgG titers of piglets. All vaccine groups produced piglets with IgG titers greater than the unvaccinated controls. The titers of piglets from the supernatant vaccine and the cell pellet vaccine were not significantly different.

### Example 6

### PEDV Nucleocapsid Protein Epitopes

In order to determine epitopes for use in PEDV immunogenic compositions for prevention and diagnosis, the following experiment was done. A purified peptide library containing 109 overlapping biotinylated peptides and covering the complete nucleocapsid (N) protein sequence of isolate USA/Colorado/2013 PEDV (GenBank Accession no. KF 272920) was produced. This peptide library was used in a PEPSCAN™ assay to identify linear antigenic epitopes in the N protein.

Twenty serum samples were obtained from a PEDV-positive pig farm in Ontario. All these samples were tested in ELISA using purified recombinant S1 and N proteins and found to be positive. Average ELISA titer against S1 was 1360, and average titer against N was 646. Five serum samples with the highest titer against N were used in the PEPSCAN assay. Three epitopes were identified on the N protein sequence that were consistently recognized by immune sera of all five pigs as follows:

(1) an epitope in the N protein at amino acid positions 285-304 (PKGENSVAACFGPRGGFKNF, SEQ ID NO:28);

(2) an epitope in the N protein at amino acid positions 257-280 (GKNTPKKNKSRATSKERDLKDIPE, SEQ ID NO:29); and

(3) an epitope in the N protein at amino acid positions 393-412 (TTQQLNEEAIYDDVGVPSDV, SEQ ID NO:30),

all numbered relative to SEQ ID NO:27 (N protein of isolate USA/Colorado/2013 PEDV; GenBank Accession no. KF272920).

In particular, to determine linear antigenic regions in the N protein, the PEPSCAN™ technique (Geysen et al., Proc. Natl. Acad. Sci. USA (1984) 81:3998-4002) was used. To this end, sets of biotinylated overlapping dodecapeptides with an offset of 4 and an overlap of 8 amino acids were designed based on the entire protein sequence of the PEDV N protein (strain USA/Colorado/2013). Individual peptides were added into triplicate wells of streptavidin-coated 96-well plates. Convalescent sera from 5 pigs, tested positive for N-specific antibodies, were incubated on the peptide-coated plates. Sera were diluted 1/100. After incubation with test serum, plates were washed and incubated with the optimal dilution of peroxidase-conjugated anti-pig polyclonal antibodies. After washings, plates were developed with a substrate solution of tetramethylbenzidine and H₂O₂. The reaction was stopped after 10 min with 1 M H₂SO₄, and the optical density at 450 nm (OD₄₅₀) was measured using an ELISA reader.

Serum from a healthy pig was included as a negative control. OD₄₅₀ values obtained with test samples at a certain peptide were expressed relative to the OD₄₅₀ value obtained with the negative control serum at the same peptide (OD₄₅₀ sample/negative, OD450 s/n). The mean OD450 s/n over all peptides within the protein was calculated, and if the OD450 s/n at a certain peptide was more than 2 times the mean over all peptides within the protein, the signal was considered specific.

Thus, methods for treating, preventing and diagnosing PEDV infection are described, as well as compositions for use with the methods. Although preferred embodiments of the subject invention have been described in some detail, it is understood that obvious variations can be made without departing from the spirit and the scope of the invention as defined by the claims.

1. A composition comprising an inactivated or attenuated Porcine Epidemic Diarrhea Virus (PEDV); a pharmaceutically acceptable vehicle; and an immunological adjuvant selected from (a) alum or (b) an adjuvant composition comprising a host defense peptide, an immunostimulatory sequence and a polyphosphazine.

2. The composition of claim 1, wherein the immunological adjuvant is alum.

3. The composition of claim 1, wherein the immunological adjuvant is an adjuvant composition comprising a host defense peptide, an immunostimulatory sequence and a polyphosphazine.

4. The composition of claim 3, wherein the polyphosphazine is selected from poly [di(sodium carboxylatophenoxy)phosphazene] (PCPP), poly(di-4-oxyphenylproprionate)phosphazene (PCEP), or a PCPP polymer comprising 90% PCPP copolymer with 10% hydroxyl groups (90:10 PCPP).

5. The composition of claim 3, wherein the immunostimulatory sequence is poly (I:C).

6. The composition of claim 1, wherein the genomic cdna sequence of the PEDV has at least 90% sequence identity to SEQ ID NO:1.

7. The composition of claim 1, wherein the attenuated PEDV comprises a mutation in a sequence of amino acids corresponding to SEQ ID NOS:28, 29 and/or 30.

8. A composition comprising:
(a) at least one isolated immunogen comprising an epitope from a PEDV spike (S) protein, a PEDV orf3 protein, a PEDV envelope (E) protein, a PEDV membrane (M) protein, and/or a PEDV nucleocapsid (N) protein;
(b) a pharmaceutically acceptable vehicle; and
(c) an immunological adjuvant.

9. The composition of claim 8, wherein the isolated immunogen is an isolated PEDV nucleocapsid immunogen.

10. The composition of claim 9, wherein the immunogen comprises the sequence of amino acids of SEQ ID NOS:28, 29 and/or 30, or the corresponding sequence from a non-USA/Colorado/2013 PEDV isolate.

11. A method of treating or preventing PEDV infection in a porcine subject or in a piglet born to a female porcine subject, comprising administering to said porcine subject a therapeutically effective amount of a composition according to claim 1.

12. The method of claim 11, wherein the porcine subject is a pregnant sow and the composition is administered to the sow prior to farrowing.

13. A method of making a PEDV composition comprising;
(a) inactivating or attenuating a PEDV; and
(b) combining the inactivated PEDV with a pharmaceutically acceptable vehicle; and an immunological adjuvant selected from (i) alum or (ii) an adjuvant composition comprising a host defense peptide, an immunostimulatory sequence and a polyphosphazine.

14. The method of claim 13, wherein the PEDV is inactivated using beta-propiolactone.

15. A method of making a PEDV composition comprising;
(a) providing at least one isolated immunogen comprising an epitope from a PEDV spike (S) protein, a PEDV orf3 protein, a PEDV envelope (E) protein, a PEDV membrane (M) protein, and/or a PEDV nucleocapsid (N) protein; and
(b) combining the immunogen with a pharmaceutically acceptable vehicle; and an immunological adjuvant

16. An isolated PEDV nucleocapsid immunogen comprising at least one PEDV epitope, wherein the immunogen comprises the sequence of amino acids of SEQ ID NOS:28, 29 and/or 30, or the corresponding sequence from a non-USA/Colorado/2013 PEDV isolate.

17. Antibodies specific for an immunogen according to claim 16.

18. The antibodies of claim 17, wherein the antibodies are polyclonal.

19. The antibodies of claim 17, wherein the antibodies are monoclonal.

20. A composition comprising the antibodies of claim 17, and a pharmaceutically acceptable vehicle.

21. A method of making a composition comprising combining the antibodies of claim 17, with a pharmaceutically acceptable vehicle.

22. A method of detecting PEDV antibodies in a biological sample comprising:
(a) reacting said biological sample with an immunogen according to claim 16 under conditions which allow PEDV antibodies, when present in the biological sample, to bind to said immunogen to form an antibody/immunogen complex; and
(b) detecting the presence or absence of said complex,
thereby detecting the presence or absence of PEDV antibodies in said sample.

23. A method of detecting PEDV infection in a biological sample comprising:
(a) reacting said biological sample with antibodies according to claim 17 under conditions which allow PEDV immunogens, when present in the biological sample, to bind to said antibodies to form an antibody/immunogen complex; and
(b) detecting the presence or absence of said complex, thereby detecting the presence or absence of PEDV infection in said sample.

24. An immunodiagnostic test kit for detecting PEDV infection, said test kit comprising an immunogen according to claim 15, and instructions for conducting the immunodiagnostic test.

25-26. (canceled)

27. An immunodiagnostic test kit for detecting PEDV infection, said test kit comprising antibodies according to claim 17, and instructions for conducting the immunodiagnostic test.


SEQUENCE LISTING (30 sequences)

SEQ ID NO: 1 - DNA, 28038 bp, Porcine epidemic diarrhea virus
  cDNA genomic sequence for isolate USA/Colorado/2013 PEDV
acttaaaaagattttctatctacggatagttagctctttttctagactcttgtctactcaattcaactaaacgaaattttgtccttccggccgcatgtccatgctgctggaagctgacgtggaatttcattaggtttgcttaagtagccatcgcaagtgctgtgctgtcctctagttcctggttggcgttccgtcgccttctacatactagacaaacagccttcctccggttccgtctgggggttgtgtggataactagttccgtctagtttgaaactagtaactgtcggctatggctagcaaccatgttacattggcttttgccaatgatgcagaaatttcagcttttggcttttgcactgctagtgaagccgtctcatactattctgaggccgccgctagtggatttatgcaatgccgtttcgtgtccttcgatctcgctgacactgttgagggattgcttcccgaagactatgtcatggtggtggtcggcactaccaagcttagtgcgtatgtggacacttttggtagccgccccaaaaacatttgtggttggctgttattttctaactgtaattacttcctcgaagagttagagcttacttttggtcgtcgtggtggtaacatcgtgccagttgaccaatacatgtgtggcgctgacggtaaacctgttcttcaggaatccgaatgggagtatacagatttctttgctgactccgaagacggtcaactcaacattgctggtatcacttatgtgaaggcctggattgtagagcgatcggatgtctcttatgcgagtcagaatttaacatctattaagtctattacttactgttcaacctatgagcatacttttcctgatggtactgccatgaaggttgcacgtactccaaagattaagaagactgttgtcttgtctgagccacttgctactatctacagggaaattggttctccttttgtggataatgggagcgatgctcgttctatcattaagagaccagtgttcctccacgcttttgttaagtgtaagtgtggtagttatcattggactgttggtgattggacttcctatgtctccacttgctgtggctttaagtgtaagccagtccttgtggcttcatgctctgctacgcctggttctgttgtggttacgcgcgctggtgctggcactggtgttaagtattacaacaacatgttcctgcgccatgtggcagacattgatgggttggcattctggcgaattctcaaggtgcagtccaaagacgacctcgcttgctctggtaaattccttgaacaccatgaggaaggtttcacagatccttgctactttttgaatgactcgagcattgctactaagctcaagtttgacatccttagtggcaagttttctgatgaagtcaaacaagctatctttgctggtcatgttgttgttggcagcgcgctcgttgacattgttgacgatgcactgggacagccttggtttatacgtaagcttggtgaccttgcaagtgcagcttgggagcagcttaaggctgtcgttagaggccttaacctcctgtctgatgaggtcgtgctctttggcaaaagacttagctgtgccactcttagtatcgttaacggtgtttttgagttcatcgccgaagtgcctgagaagttggctgcggctgttacagtttttgtcaacttcttgaatgagctttttgagtctgcctgtgactgcttaaaggtcggaggtaaaacctttaacaaggttggctcttatgttctttttgacaacgcattggttaagcttgtcaaggcaaaagttcgcggcccacgacaggcaggtgtttgtgaagttcgttacacaagccttgttattgggagtactaccaaggtggtttccaagcgcgttgaaaatgccaatgtgaatctcgtcgtcgttgacgaggatgtgaccctcaacaccactggtcgtacagttgttgttgacggacttgcattcttcgagagtgacgggttttacagacatcttgctgatgctgacgttgtcattgaacatcctgtttataagtctgcttgtgagctcaagccagtttttgagtgtgacccaatacctgattttcctatgcctgtggccgctagtgttgcagagctttgtgtgcaaactgatctgttgcttaaaaattacaacactccttataaaacttacagctgcgttgtgagaggtgataagtgttgtatcacttgcaccttacatttcacagcaccaagttatatggaggctgctgctaattttgtagacctctgtaccaagaacattggtactgctggttttcatgagttttacattacggcccatgaacaacaggatctgcaagggttcgtaaccacttgttgcacgatgtcaggttttgagtgttttatgcctataatcccacagtgtccagcagtgcttgaagagattgatggtggtagcatctggcggtcttttatcactggtcttaatacaatgtgggatttttgcaagcatcttaaagtcagctttggactagatggcattgttgtcactgtagcacgcaaatttaaacgacttggtgctctcttggcagaaatgtataacacttacctttcaactgtggtggaaaacttggtactggccggtgttagcttcaagtattatgccaccagtgtcccaaaaattgttttgggctgttgttttcacagtgttaaaagtgttcttgcaagtgccttccagattcctgtccaggcaggcgttgagaagtttaaagtcttccttaactgtgttcaccctgttgtaccacgtgtcattgaaacttcttttgtggaattagaagagacgacatttaaaccaccagcactcaatggtagtattgctattgttgatggctttgctttctattatgatggaacactatactatcccaccgatggtaatagcgttgttcctatctgctttaagaagaaaggtggtggtgatgtcaaattctctgatgaagtctctgttaaaaccattgacccagtttataaggtctcccttgaatttgagttcgagtctgagactattatggctgtgcttaataaggctgttggtaattgtatcaaggttacaggtggttgggacgatgttgttgagtatatcaatgttgccattgaggttcttaaagatcacatcgatgtgcctaagtactacatctatgatgaggaaggtggcaccgatcctaatctgcccgtaatggtttctcagtggccgttgaatgatgacacgatctcacaggatctgcttgatgttgaagttgttactgatgcgccagttgatttcgagggtgatgaagtagactcctctgaccctgataaggtggcagacgtggctaactctgagcctgaggatgacggtcttaatgtagctcctgaaacaaatgtagagtctgaagttgaggaagttgccgcaaccttgtcctttattaaagatacaccttccacagttactaaggatccttttgcttttgactttgcaagctatggaggacttaaggttttaagacaatctcataacaactgctgggttacttctaccttggtgcagctacaattgcttggcatcgttgatgaccctgcaatggagctttttagtgctggtagagttggtccaatggttcgcaaatgctatgagtcacaaaaggctatcttgggatctttgggtgatgtgtcggcttgcctagagtctctgactaaggacctacacacacttaagattacctgttctgtagtctgtggttgtggtactggtgaacgtatctatgatggttgtgcttttcgtatgacgccaactttggaaccgttcccatatggtgcttgtgctcagtgtgctcaagttttgatgcacacttttaaaagtattgttggcaccggcatcttttgtcgagatactactgctctctccttggattctttggttgtaaaacctctttgtgcggctgcttttataggcaaggatagtggtcattatgtcactaacttttatgatgctgctatggctattgatggttatggtcgtcatcagataaagtatgacacactgaacactatttgtgttaaagacgttaattggacagcaccttttgtcccagacgttgagcctgtattggagcctgttgtcaaacctttctattcttataagaatgttgatttttaccaaggagattttagtgaccttgttaaacttccatgtgattttgttgttaatgctgcaaatgagaatttgtctcacggtggcggcatagcaaaggccattgatgtttataccaagggcatgttgcagaagtgctcgaatgattacattaaagcacacggtcccattaaagttggacgtggtgtcatgttggaggcattaggtcttaaggtctttaatgttgttggtccacgtaagggtaagcatgcacctgagcttcttgttaaggcttataagtccgtttttgctaattcaggtgttgctcttacacctttgattagtgttggaatttttagtgttcctttggaagaatctttatctgcttttcttgcatgtgttggtgatcgccactgtaagtgcttttgttatagtgacaaagagcgcgaggcgatcattaattacatggatggcttggtagatgctattttcaaagatgcacttgttgatactactcctgtccaggaagatgttcaacaagtttcacaaaaaccagttttgcctaattttgaacctttcaggattgaaggtgctcatgctttctatgagtgcaaccctgaaggtttgatgtcattaggtgctgacaagctggtgttgtttacaaattccaatttggatttttgtagcgttggtaagtgtcttaacaatgtgactggcggtgcattgcttgaagccataaatgtatttaaaaagagtaacaaaacagtgcctgctggcaactgtgttacttttgagtgtgcagatatgatttctattactatggtagtattgccatctgacggtgatgctaattatgacaaaaattatgcacgcgccgtcgtcaaggtatctaagcttaaaggcaagttattgcttgctgttggtgatgccatgttgtattccaagttgtcccacctcagcgtgttaggtttcgtatccacacctgatgatgtggagcgtttctacgcaaataagagtgtggttattaaagttactgaggatacacgtagtgttaagactgttaaagtagaatccactgttacttatggacaacaaattggaccttgtcttgttaatgacaccgttgtcacagacaacaaacctgttgttgctgatgttgtagctaaggttgtaccaagtgctaattgggattcacattatggttttgataaggctggtgagttccacatgctagaccatactgggtttgcctttcctagtgaagttgttaacggtaggcgtgtgcttaaaaccacagataataactgttgggttaatgttacatgtttacaattacagtttgctagatttaggttcaagtcagcaggtctacaggctatgtgggagtcctattgtactggtgatgttgctatgtttgtgcattggttgtactggcttactggtgttgacaaaggtcagcctagtgattcagaaaatgcacttaacatgttgtctaagtacattgttcctgctggttctgtcactattgaacgtgtcacgcatgacggttgttgttgtagtaagcgtgttgtcactgcaccagttgtgaatgctagcgtgttgaagcttggcgtcgaggatggtctttgtccacatggtcttaactacattgacaaagttgttgtagttaaaggtactacaattgttgtcaatgttggaaaacctgtagtggcaccatcgcacctctttcttaagggtgtttcctacacaacattcctagataatggtaacggtgttgccggccattatactgtttttgatcatgacactggtatggtgcatgatggagatgtttttgtaccaggtgatctcaatgtgtctcctgttacaaatgttgtcgtctcagagcagacggctgttgtgattaaagaccctgtgaagaaagtagagttagacgctacaaagctgttagacactatgaattatgcatcggaaagattcttttcctttggtgattttatgtcacgtaatttaattacagtgtttttgtacatccttagtattttgggtctctgttttagggcctttcgtaagagggatgttaaagttctagctggtgtaccccaacgtactggtattatattgcgtaaaagtgtgcgctataatgcaaaggctttgggtgtcttcttcaagctaaaactttattggttcaaagttcttggtaagtttagtttgggtatttatgcattgtatgcattactattcatgacaatacgctttacacctataggtggccctgtttgtgatgatgttgttgctggttatgctaattctagttttgacaagaatgagtattgcaacagtgttatttgtaaggtctgtctctatgggtaccaggaactttcggacttctctcacacacaggtagtatggcaacaccttagagacccattaattggtaatgtgatgcctttcttttatttggcatttctggcaatttttgggggtgtttatgtaaaggctattactctctattttattttccagtatcttaacatacttggtgtgtttttgggcctacaacagtccatttggtttttgcagcttgtgccttttgatgtctttggtgacgagatcgtcgtctttttcatcgttacacgcgtattgatgttccttaagcatgttttccttggctgcgataaggcatcttgtgtggcttgctctaagagtgctcgccttaagcgcgttcctgtccagactatttttcagggtactagcaaatccttctacgtacatgccaatggtggttctaagttctgtaagaagcacaatttcttttgtttaaattgtgattcttatggtccaggctgcacttttattaatgacgtcattgcaactgaagttggtaatgttgtcaaacttaatgtgcaaccgacaggtcctgccactattcttattgacaaggttgaattcagtaatggtttttactatctttatagtggtgacacattttggaagtacaactttgacataacagataacaaatacacttgcaaagagtcacttaaaaattgtagcataatcacagactttattgtttttaacaataatggttccaatgtaaatcaggttaagaatgcatgtgtgtatttttcacagatgctttgtaaacctgttaagttagtggactcagcgttgttggccagtttgtctgttgattttggtgcaagcttacatagtgcttttgttagtgtgttgtcgaatagttttggcaaagacctgtcaagttgtaatgacatgcaggattgcaagagcacattgggttttgatgatgtaccattggatacctttaatgctgctgttgctgaggctcatcgttacgatgtcctcttgactgacatgtcgttcaacaattttaccaccagttatgcaaaaccagaggaaaaacttcccgtccatgacattgccacgtgtatgcgtgtaggtgccaagattgttaatcataacgttcttgtcaaggatagtatacctgtggtgtggcttgtacgtgatttcattgccctttctgaagaaactaggaagtacattattcgtacgactaaagttaagggtataaccttcatgttgacctttaatgattgtcgtatgcatactaccatacctactgtttgcattgcaaataagaagggtgcaggtcttcctagtttttcaaaggttaagaaattcttctggtttttgtgtctgttcatagttgctgttttctttgcactaagcttttttgattttagtactcaggttagcagtgatagtgattatgacttcaagtatattgagagtggccagttgaagacttttgacaatccacttagttgtgtgcataatgtctttagtaacttcgaccagtggcatgatgccaagtttggtttcacccccgtcaacaatcctagttgtcctatagtcgttggtgtatcagacgaagcgcgcactgttccaggtatcccagcaggtgtttatttagctggtaaaacacttgtttttgctattaacaccatttttggtacatctggtttgtgctttgatgctagtggcgttgctgataagggcgcttgcatttttaattcggcttgcaccacattatctggtttgggtggaactgctgtctactgttataagaatggtctagttgaaggtgctaaactttatagtgagttggcacctcatagctactataaaatggtagatggtaatgctgtgtctttacctgaaattatctcacgcggctttggcatccgtactatccgtacaaaggctatgacctactgtcgcgttggccagtgtgtgcaatctgcagaaggtgtttgttttggcgccgatagattctttgtctataatgcagaatctggttctgactttgtttgtggcacagggctctttacattgttgatgaacgttattagtgttttttccaagacagtaccagtaactgtgttgtctggtcaaatactttttaattgcattattgcttttgctgctgttgcggtgtgtttcttatttacaaagtttaagcgcatgttcggtgatatgtctgttggcgttttcactgtcggtgcttgtactttgttgaacaatgtttcctacattgtaacacagaacacacttggcatgttgggctatgcaactttgtactttttgtgcactaaaggtgttagatatatgtggatttggcatttgggatttttgatctcatatatacttattgcaccatggtgggttttgatggtttatgccttttcagccatttttgagtttatgcctaacctttttaagcttaaggtttcaacacaactttttgagggtgacaagttcgtaggctcttttgaaaatgctgcagcaggtacatttgtgcttgatatgcatgcctatgagagacttgccaactctatctcaactgaaaaactgcgtcagtatgctagtacttacaataagtacaagtattattcaggcagtgcttcagaggctgattacaggcttgcttgttttgcccatttggccaaggctatgatggattatgcttctaatcacaacgacacgttatacacaccacccactgtgagttacaattcaactctacaggctggcttgcgtaagatggcacaaccatctggtgttgttgagaagtgcatagttcgtgtttgctatggtaatatggctcttaatggcctatggcttggtgatactgttatctgcccacgccatgttatagcgtctagtactactagcactatagattatgactatgccctttctgttttacgcctccacaacttctccatttcatctggtaatgttttcctaggtgttgtgggtgtaaccatgcgaggtgctttgttgcagataaaggttaatcaaaacaatgtccacacgcctaagtacacctatcgcacagttagaccgggtgaatcttttaatatcttggcgtgctatgatggttctgcagctggtgtttacggcgttaacatgcgctctaattacactattagaggctcgttcattaatggcgcttgtggttcacctggttataacattaacaatggtaccgttgagttttgctatttacaccagcttgaacttggttcaggctgtcatgttggtagcgacttagatggtgttatgtatggtggttatgaggaccaacctactttgcaagttgaaggcgctagtagtctgtttacagagaatgtgttggcatttctttatgcagcactcattaatggttctacctggtggcttagttcttctaggattgctgtagacaggtttaatgagtgggctgttcataatggtatgacaacagtagttaatactgattgcttttctattcttgctgctaagactggtgttgatgtacaacgtttgttggcctcaatccagtctctgcataagaattttggtggaaagcaaattcttggctatacctcgttgacagatgagtttactacaggtgaagttatacgtcaaatgtatggcgttaatcttcagagtggttatgtttcacgcgcctgtagaaatgtcttgctggttggttcttttctgactttcttttggtcagaattagtttcctacactaagttcttttgggtaaatcctggttatgtcacacctatgtttgcgtgtttgtcattgctgtcctcacttttgatgttcacactcaagcataagacattgtttttccaggtctttctaatacctgctctgattgttacatcttgcattaatttggcatttgatgttgaagtctacaactatttggcagagcattttgattaccatgtttctctcatgggttttaatgcacaaggtcttgttaacatctttgtctgctttgttgttaccattttacacggcacatacacatggcgcttttttaacacacctgtgagttctgtcacttatgtggtagctttgctgactgcggcatataactatttttacgctagtgacattcttagttgtgctatgacactatttgctagtgtgactggcaactggttcgttggtgctgtttgttataaagctgctgtttatatggccttgagatttcctacttttgtggctatttttggtgatattaagagtgttatgttctgttaccttgtgttgggttattttacctgttgcttctacggtattctctactggttcaacaggttttttaaggttagtgtaggtgtctatgactatactgttagtgctgctgagtttaagtatatggttgctaacggcctacgtgcaccaactggaacacttgattcactacttctgtctgccaaattgattggtattggtggtgagcggaatattaagatttcttccgttcagtctaaactgactgatattaagtgtagtaacgttgtgcttttaggctgtctctctagcatgaatgtctcagcaaattcaacagaatgggcctattgtgttgacttgcataacaagatcaacttgtgtaatgacccagaaaaagcgcaggaaatgctacttgctttgttggcatttttccttagtaagaatagtgcttttggtttagatgacttattggaatcctattttaatgacaatagtatgttgcagagtgttgcatctacttatgtcggtttgccttcttatgtcatttatgaaaatgcacgccaacagtatgaagatgctgttaataatggttctccacctcagttggttaagcaattgcgccatgccatgaatgtagcaaagagcgaatttgaccgtgaggcttctactcagcgtaagcttgatagaatggcggaacaggctgcagcacagatgtacaaagaggcacgagcagttaataggaagtccaaagttgtaagtgctatgcattcactgctttttggtatgttgagacgtttggacatgtcttctgtagacaccattctcaacttggcaaaggatggggttgtacctctgtctgtcataccggcagtcagtgctactaagcttaacattgttacttctgatatcgattcttataatcgtatccagcgtgagggatgtgtccactacgctggtaccatttggaatataattgatatcaaggacaatgatggcaaggtggtacacgttaaggaggtaaccgcacagaatgctgagtccctgtcatggcccctggtccttgggtgtgagcgtattgtcaagctccagaataatgaaattattcctggtaagctgaagcagcgctccattaaggcagaaggagatggcatagttggagaaggtaaggcactttacaataatgagggtggacgtacttttatgtatgctttcatctcggacaaaccggacctgcgtgtagtcaagtgggagttcgatggtggttgtaacactattgagctagaaccaccacgtaagttcttggtggattctcctaatggtgcacagatcaagtatctctactttgttcgtaaccttaacacgttacgtaggggtgctgttctcggctacataggtgccactgtacgcttgcaggctggtaaacaaacagaacaggctattaactcttcattgttgacactttgcgctttcgctgtggatcctgctaagacctacatcgatgctgtcaaaagtggtcacaaaccagtaggtaactgtgttaagatgttggccaatggttctggtaatggacaagctgttactaatggtgtggaggctagtactaaccaggattcatacggtggtgcgtccgtgtgtctatattgtagagcacatgttgagcatccatctatggatggtttttgcagactgaaaggcaagtacgtacaggttccactaggtacagtggatcctatacgttttgtacttgagaatgacgtttgcaaggtttgtggttgttggctggctaatggctgcacttgtgacagatccattatgcaaagcactgatatggcttatttaaacgagtacggggctctagtgcagctcgactagagccmtgtaacggtactgatacacaacatgtgtatcgtgcttttgacatctacaacaaggatgttgcttgtctaggtaaattcctcaaggtgaactgtgttcgcctgaagaatttggataagcatgatgcattctatgttgtcaaaagatgtaccaagtctgcgatggaacacgagcaatccatctatagcagacttgaaaagtgtggagccgtagccgaacacgatttcttcacttggaaggatggtcgtgccatctatggtaacgtttgtagaaaggatcttaccgagtatactatgatggatttgtgttacgctttacgtaactttgatgaaaacaattgcgatgttcttaagagcattttaattaaggtaggcgcttgtgaggagtcctacttcaataataaagtctggtttgaccctgttgaaaatgaagacattcatcgtgtctatgcattgttaggtaccattgtttcacgtgctatgcttaaatgcgttaagttctgtgatgcaatggttgaacaaggtatagttggtgttgtcacattagataatcaggatcttaatggtgatttttatgattttggtgattttacttgtagcatcaagggaatgggtatacccatttgcacatcatattactcttatatgatgcctgttatgggtatgactaattgccttgctagtgagtgttttgttaagagtgatatatttggtgaggatttcaagtcatatgacctgctggaatatgatttcacggagcataagacagcactcttcaacaagtatttcaagtattggggactgcaataccaccctaactgtgtggactgcagtgatgagcagtgcatagttcactgtgccaacttcaatacgttgttttccactactatacctattacggcatttggacctttgtgtcgcaagtgttggattgatggtgttccactggtaactacagctggttatcattttaaacagttaggtatagtttggaacaatgacctcaacttacactctagcaggctctctattaacgaattactccagttttgtagtgatcctgcattgcttatagcatcatcaccagcccttgttgatcagcgtactgtttgcttttcagttgcagcgctaggtacaggtatgactaaccagactgttaaacctggccatttcaataaggagttttatgacttcttacttgagcaaggtttcttttctgagggctctgagcttactttaaagcacttcttctttgcacagaagggtgatgcagctgttaaggattttgactactataggtataatagacctactgttctggacatttgccaagctcgcgtcgtgtatcaaatagtgcaacgctattttgatatttacgaaggtggttgtatcactgctaaagaggtggttgttacaaaccttaacaagagcgcaggttatcctttgaacaagtttggtaaagctggtctttactatgagtctttatcctatgaggaacaggatgaactttatgcttatactaagcgtaacatcctgcccactatgacacagctcaaccttaaatatgctataagtggcaaagaacgtgcacgcacagtgggtggtgtttcgcttttgtcaaccatgactactcggcagtatcatcagaaacaccttaagtccatagttaatactaggggcgcttcggttgttattggtactactaagttttatggtggttgggacaatatgcttaagaaccttattgatggtgttgaaaatccgtgtcttatgggttgggactacccaaagtgcgacagagcactgcccaatatgatacgtatgatttcagccatgattttaggctctaagcacaccacatgctgcagttccactgaccgctttttcaggttgtgcaatgaattggctcaagtccttactgaggttgtttattctaatggaggtttttatttgaagccaggtggtactacctctggtgatgcaaccaccgcatatgcaaactcagtttttaatatcttccaagcagtaagtgccaatgttaacaaacttcttagtgttgacagcaatgtctgtcataatttagaagttaagcaattgcagcgtaagctttatgagtgctgttatagatcaactaccgtcgatgaccagttcgtcgttgagtattatggttacttgcgtaaacatttttcaatgatgattctttctgatgatggcgttgtttgttataacaatgactatgcatcacttggttatgtcgctgatcttaacgcattcaaggctgttttgtattaccagaacaatgtcttcatgagcgcctctaaatgttggatcgagcctgacattaataaaggtcctcatgaattttgctcgcagcatactatgcagattgtcgataaagatggtacttattaccttccttaccctgatccttcaagaattctctctgcaggtgtgtttgttgatgacgttgttaaaactgatgcagttgtattgcttgaacgttatgtgtcattggctatagatgcctacccgttatctaagcatgaaaaccctgaatataagaaggtgttttatgtgcttttggattgggttaagcatctgtacaaaactcttaatgctggtgtgttagagtctttttctgtcacacttttggaagattctactgctaaattctgggatgagagcttttatgccaacatgtatgagaaatctgcagttttacaatctgcagggctttgtgttgtttgtggctctcaaactgttttacgttgtggtgattgtctacggcgtcctatgctttgtactaagtgtgcttatgatcatgtcattggaacaactcacaagttcattttggccatcactccatatgtgtgttgtgcttcagattgtggtgtcaatgatgtaactaagctctacttaggtggtcttagttattggtgtcatgaccacaagccacgtcttgcattcccgttgtgctctgctggtaatgtttttggcttgtacaaaaattctgctaccggctcacccgatgttgaagactttaatcgcattgctacatccgattggactgatgtttctgactacaggttggcaaatgatgtcaaggactcattgcgtctgtttgcagcggaaactatcaaggccaaggaggagagcgttaagtcatcctatgcttgtgcaacactacatgaggttgtaggacctaaagagttgttgctcaaatgggaagtcggcagacccaaaccaccccttaatagaaattcggttttcacttgttatcatataacgaagaacaccaaatttcaaatcggtgagtttgtgtttgagaaggcagaatatgataatgatgctgtaacatataaaactaccgccacaacaaaacttgttcctggcatggtttttgtgcttacctcacataatgttcagccattgcgcgcaccgaccattgctaatcaagaacgttattccactatacataagttgcatcctgcttttaacatacctgaagcttattctagcttagtgccctattaccaattgattggtaagcagaagattacaactattcagggacctcccggtagtggtaaatctcactgtgttatagggctaggtttgtactatccaggtgcacgtatagtgtttacagcttgttctcatgcagcggtcgattcactttgtgtgaaagcttccactgcttatagcaatgacaaatgttcacgcatcataccacagcgcgctcgtgttgagtgttatgatggtttcaagtctaataatactagtgctcagtaccttttctctactgtcaatgctttgccagagtgcaatgcggacattgttgtggtggatgaggtctctatgtgcactaattatgacttgtctgtcataaatcagcgcatcagctataggcatgtagtctatgttggtgaccctcaacagctgcctgcaccacgtgttatgatttcacgtggtactttggaaccaaaggactacaacgttgtcactcaacgcatgtgtgcccttaagcctgatgttttcttgcacaagtgttatcgctgtcctgctgagatagtgcgtactgtgtctgagatggtctatgaaaaccaattcattcctgtgcacccagatagcaagcagtgttttaaaatcttttgcaagggtaatgttcaggttgataatggttcaagcattaatcgcaggcaattggatgttgtgcgtatgtttttggctaaaaatcctaggtggtcaaaggctgtttttatttctccttataacagccagaattatgttgccagccgcatgctaggtctacaaattcagacagttgactcatcccagggtagtgagtatgactatgtcatttacacacaaacttcagatactgcccatgcctgtaatgttaacaggtttaatgttgccatcacaagggccaagaaaggcatattatgtataatgtgcgataggtccctttttgatgtgcttaaattctttgagcttaaattgtctgatttgcaggctaatgagggttgtggtctttttaaagactgtagcagaggtgatgatctgttgccaccatctcacgctaacaccttcatgtctttagcggacaattttaagactgatcaagatcttgctgttcaaataggtgttaatggacccattaaatatgagcatgttatctcgtttatgggtttccgttttgatatcaacatacccaaccatcatactctcttttgcacacgcgactttgccatgcgcaatgttagaggttggttaggctttgacgttgaaggagcacatgttgttggctctaacgtcggtacaaatgtcccattgcaattagggttttctaacggtgttgattttgttgtcagacctgaaggttgcgttgtaacagagtctggtgactacattaaacccgtcagagctcgtgctccaccaggggaacaattcgcacaccttttgcctttacttaaacgcggccaaccatgggatgttgtccgcaaacgtatagtgcagatgtgtagtgactacctggccaacctatcagacatactaatttttgtgttgtgggctggtggtttggagttgacaactatgcgttattttgtcaagattggaccaagtaagagttgtgattgtggtaaggttgctacttgttacaatagtgcgctgcatacgtactgttgtttcaaacatgcccttggttgtgattatctgtataacccatactgtattgatatacagcagtggggatacaagggatcacttagccttaaccaccatgagcattgtaatgtacatagaaacgagcatgtggcttctggtgatgccataatgactcgctgtctggccatacatgattgctttgtcaagaacgttgactggtccatcacatacccatttattggtaatgaggctgttattaataagagcggccgaattgtgcaatcacacactatgcggtcagttcttaagttatacaatccgaaagccatatatgatattggcaatcctaagggcattagatgtgccgtaacggatgctaagtggttttgctttgacaagaatcctactaattctaatgtcaagacattggagtatgactatataacacatggccaatttgatgggttgtgcttgttttggaattgcaatgtagacatgtatccagaattttctgtggtctgtcgttttgatactcgctgtaggtcaccactcaacttggagggttgtaatggtggttcactgtatgttaataatcatgcattccatacaccggcttttgacaagcgtgcttttgctaagttgaagccaatgccatttttcttttatgatgatactgagtgtgacaagttacaggactccataaactatgttcctcttagggctagtaactgcattactaaatgtaatgttggtggtgctgtctgtagtaagcattgtgctatgtatcatagctatgttaatgcttacaacacttttacgtcggcgggctttactatttgggtgcctacttcgtttgacacctataatctgtggcagacatttagtaacaatttgcaaggtcttgagaacattgctttcaatgtcgtaaagaaaggatcttttgttggtgccgaaggtgaacttcctgtagctgtggttaatgacaaagtgctcgttagagatggtactgttgatactcttgtttttacaaacaagacatcactacccactaacgtagcttttgagttgtatgccaagcgtaaggtaggactcaccccacccattacgatcctacgtaacttgggtgtagtttgtacatctaagtgtgtcatttgggactatgaagccgaacgtccacttactacttttacaaaggatgtttgtaaatataccgactttgagggtgacgtctgtacactctttgataacagcattgttggttcattagagcgattctccatgacccaaaatgctgtgcttatgtcacttacagctgttaaaaagcttactggcataaagttaacttatggttatcttaatggtgtcccagttaacacacatgaagataaaccttttacttggtatatttacactaggaagaacggcaagttcgaggaccatcctgatggctattttacccaaggtagaacaaccgctgattttagccctcgtagcgacatggaaaaggacttcctaagtatggatatgggtctgtttattaacaagtacggacttgaagattacggctttgagcacgttgtgtatggtgatgtttcaaaaaccacccttggtggtttgcatctactaatttcgcaggtgcgtctggcctgtatgggtgtgctcaaaatagacgagtttgtgtctagtaatgatagcacgttaaagtcttgtactgttacatatgctgataaccctagtagtaagatggtttgtacgtatatggatctcctgcttgacgattttgtcagcattcttaaatctttggatttgggcgttgtatctaaagttcatgaagttatggtcgattgtaaaatgtggaggtggatgttgtggtgtaaggatcataaactccagacattttatccgcaacttcaggccagtgaatggaagtgtggttattccatgccttctatttacaagatacaacgtatgtgtttagaaccttgcaatctctacaactatggtgctggtattaagttacctgatggcattatgtttaacgtagttaaatacacacagctttgtcaatatctcaatagcaccacaatgtgtgtaccccatcacatgcgtgtgctacatcttggtgctggctccgacaagggtgttgcacctggcacggctgtcttacgacgttggttgccactggatgccattatagttgacaatgatagtgtggattacgttagcgatgctgattatagtgttacaggagattgctctaccttatacctgtcagataagtttgatttagttatatctgatatgtatgatggtaagattaaaagttgtgatggggagaacgtgtctaaagaaggcttctttccctatattaatggtgtcatcaccgaaaagttggcacttggtggtactgtagctattaaggtgacggagtttagttggaataagaagttgtatgaactcattcagaggtttgagtattggacaatgttctgtaccagtgttaacacgtcatcgtcagaggcattcttaattggtgttcactatttaggtgattttgcaagtggcgctgtgattgacggcaacactatgcatgccaattatatcttctggcgtaattccacaattatgactatgtcttacaatagtgtacttgatttaagcaagttcaattgtaagcataaggctacagttgtcattaatttaaaagattcatccattagtgatgttgtgttaggtttgttgaagaatggtaagttgctagtgcgtaataatgacgccatttgtggtttttctaatcatttggtcaacgtaaacaaatgaagtctttaacctacttctggttgttcttaccagtactttcaacacttagcctaccacaagatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaaaacattgggccccactgctaataatgatgttacaacaggtcgtaattgcctatttaacaaagccatcccagctcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacacctctgtcattcttgctgaaggctcaattgtacttcatactgctttaggaacaaatttttcttttgtttgcagtaattcctcaaatcctcacttagccaccttcgccatacctctgggtgctacccaagtaccttattattgttttcttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttaccctatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttactttcaattcacaaagggtgagttgattactggcacgcctaaaccacttgaaggtgtcacggacgtttcttttatgactctggatgtgtgtaccaagtatactatctatggctttaaaggtgagggtatcattacccttacaaattctagctttttggcaggtgtttattacacatctgattctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacaactcagcgctaggcttgagtctgttgaagttaactctatgcttactatttctgaagaggctctacagttagctaccattagttcgtttaatggtgatggatataattttactaatgtgctgggtgtttctgtgtatgatcctgcaagtggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgttcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatctcagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacacttgatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccacgggttgttgtggatgctgcggctgctgctgtgcttgtttctcaggttgttgtaggggtcctagacttcaaccttacgaagtttttgaaaaggtccacgtgcagtgatgtttcttggactttttcaatacacgattgacacagttgtcaaagatgtctcaaagtctgctaacttgtctttggatgctgtccaagagttggagctcaatgtagttccaattagacaagcttcaaatgtgacgggttttcttttcaccagtgtttttatctacttctttgcactgtttaaagcgtcttctttgaggcgcaattatattatgttggcagcgcgttttgctgtcattgttctttattgcccacttttatattattgtggtgcatttttagatgcaactattatttgttgcacacttattggcaggctttgtttagtctgcttttactcctggcgctataaaaatgcgctctttattatttttaatactacgacactttctttcctcaatggtaaagcagcttattatgacggcaaatccattgtgattttagaaggtggtgaccattacatcacttttggcaactctcttgttgcttttgttagtagcatcgacttgtatctagctatacgtgggcggcaagaagctgacctacagctgttgcgaactgttgagcttcttgatggcaagaagctttatgtcttttcgcaacatcaaattgttggcattactaatgctgcatttgactcaattcaactagacgagtatgctacaattagtgaatgataatggtctagtagttaatgttatactttggcttttcgtactctttttcctgcttattataagcattactttcgtccaattggttaatctgtgcttcacttgtcaccggttgtgtaatagcgcagtttacacacctatagggcgtttgtatagagtttataagtcttacatgcaaatagaccccctccctagtactgttattgacgtataaacgaaatatgtctaacggttctattcccgttgatgaggtgattcaacaccttagaaactggaatttcacatggaatatcatactgacgatactacttgtagtgcttcagtatggccattacaagtactctgcgttcttgtatggtgtcaagatggctattctatggatactttggcctcttgtgttagcactgtcactttttgatgcatgggctagctttcaggtcaattgggtcttttttgctttcagcatccttatggcttgcatcactcttatgctgtggataatgtactttgtcaatagcattcggttgtggcgcaggacacattcttggtggtctttcaatcctgaaacagacgcgcttctcactacttctgtgatgggccgacaggtctgcattccagtgcttggagcaccaactggtgtaacgctaacactccttagtggtacattgcttgtagagggctataaggttgctactggcgtacaggtaagtcaattacctaatttcgtcacagtcgccaaggccactacaacaattgtctacggacgtgttggtcgttcagtcaatgcttcatctggcactggttgggctttctatgtccggtccaaacacggcgactactcagctgtgagtaatccgagttcggttctcacagatagtgagaaagtgcttcatttagtctaaacagaaactttatggcttctgtcagttttcaggatcgtggccgcaaacgggtgccattatccctctatgcccctcttagggttactaatgacaaacccctttctaaggtacttgcaaataatgctgtacccactaataaaggaaataaggaccagcaaattggatactggaatgagcaaattcgctggcgcatgcgccgtggtgagcgaattgaacaaccttccaattggcatttctactacctcggaacaggacctcacgccgacctccgctataggactcgtactgagggtgttttctgggttgctaaagaaggcgcaaagactgaacccactaacctgggtgtcagaaaggcgtctgaaaagccaattattccaaatttctctcaacagcttcccagcgtagttgagattgttgaacctaacacacctcctacttcacgtgcaaattcacgtagcaggagtcgtggtaatggcaacaacaggtccagatctccaagtaacaacagaggcaataaccagtcccgcggtaattcacagaatcgtggaaataaccagggtcgtggagcttctcagaacagaggaggcaataataataacaataacaagtctcgtaaccagtccaagaacagaaaccagtcaaatgaccgtggtggtgtaacatcacgcgatgatctggtggctgctgtcaaggatgcccttaaatctttgggtattggcgaaaaccctgacaagcttaagcaacagcagaagcccaaacaggaaaggtctgacagcagcggcaaaaatacacctaagaagaacaaatccagagccacttcgaaagaacgtgacctcaaagacatcccagagtggaggagaattcccaagggcgaaaatagcgtagcagcttgcttcggacccaggggaggcttcaaaaattttggagatgcggaatttgtcgaaaaaggtgttgatgcctcaggctatgctcagatcgccagtttagcaccaaatgttgcagcattgctctttggtggtaatgtggctgttcgtgagctagcggactcttacgagattacatataattataaaatgactgtgccaaagtctgatccaaatgtagagcttcttgtttcacaggtggatgcatttaaaactgggaatgcaaaaccccagagaaagaaggaaaagaagaacaagcgtgaaaccacgcagcagctgaatgaagaggccatctacgatgatgtgggtgtgccatctgatgtgactcatgccaatttggaatgggacacagctgttgatggtggtgacacggccgttgaaattatcaacgagatcttcgacacaggaaattaaacaatgtttgactggcttatcctggctatgtcccagggtagtgccattacactgttattactgagtgtttttctagcgacttggctgctgggctatggctttgccctctaactagcggtcttggtcttgcacacaacggtaagccagtggtaatgtcagtgcaagaaggatattaccatagcactgtcatgaggggaacgcagtaccttttcatctaaacctttgcacgagtaatcaaagatccgcttgacgagcctatatggaagagcgtgccaggtatttgactcaaggactgttagtaactgaagacctgacggtgttgatatggatacac

SEQ ID NO: 2 - protein, 12 aa, Artificial Sequence
  Synthetic
VQLRIRVAVIRA

SEQ ID NO: 3 - protein, 12 aa, Artificial Sequence
  Synthetic
VQRWLIVWRIRK

SEQ ID NO: 4 - protein, 12 aa, Artificial Sequence
  Synthetic
VRLIVAVRIWRR

SEQ ID NO: 5 - protein, 13 aa, Artificial Sequence
  Synthetic
ILPWKWPWWPWRR

SEQ ID NO: 6 - protein, 13 aa, Artificial Sequence
  Synthetic
ILKWKWPWWPWRR

SEQ ID NO: 7 - protein, 13 aa, Artificial Sequence
  Synthetic
ILPWKKPWWPWRR

SEQ ID NO: 8 - protein, 13 aa, Artificial Sequence
  Synthetic
ILKWKWPWWKWRR

SEQ ID NO: 9 - protein, 13 aa, Artificial Sequence
  Synthetic
ILRWKWRWWRWRR

SEQ ID NO: 10 - protein, 34 aa, Artificial Sequence
  Synthetic
  Variant (2)..(2): Xaa IS Dhb
  Variant (5)..(5): Xaa is Dha
  Variant (8)..(8): Xaa is Abu
  Variant (13)..(13): Xaa is Abu
  Variant (23)..(23): Xaa is Abu
  Variant (25)..(25): Xaa is Abu
  Variant (33)..(33): Xaa is Dha
IXAIXLAXPGAKXGALMGANMKXAXANASINVXK

SEQ ID NO: 11 - protein, 12 aa, Artificial Sequence
  Synthetic
VFLRRIRVIVIR

SEQ ID NO: 12 - protein, 12 aa, Artificial Sequence
  Synthetic
VFWRRIRVWVIR

SEQ ID NO: 13 - protein, 12 aa, Artificial Sequence
  Synthetic
VQLRAIRVRVIR

SEQ ID NO: 14 - protein, 12 aa, Artificial Sequence
  Synthetic
VQLRRIRVWVIR

SEQ ID NO: 15 - protein, 12 aa, Artificial Sequence
  Synthetic
VQWRAIRVRVIR

SEQ ID NO: 16 - protein, 12 aa, Artificial Sequence
  Synthetic
VQWRRIRVWVIR

SEQ ID NO: 17 - DNA, 20 bp, Artificial Sequence
  Synthetic
tccatgacgttcctgacgtt

SEQ ID NO: 18 - DNA, 22 bp, Artificial Sequence
  Synthetic
tcgtcgttgtcgttttgtcgtt

SEQ ID NO: 19 - DNA, 24 bp, Artificial Sequence
  Synthetic
tcgtcgttttgtcgttttgtcgtt

SEQ ID NO: 20 - DNA, 21 bp, Artificial Sequence
  Synthetic
ggggacgacgtcgtggggggg

SEQ ID NO: 21 - DNA, 22 bp, Artificial Sequence
  Synthetic
tcgtcgttttcggcgcgcgccg

SEQ ID NO: 22 - DNA, 32 bp, Artificial Sequence
  Synthetic
aaaaaaggtacctaaatagtatgtttctgaaa

SEQ ID NO: 23 - protein, 1386 aa, Porcine epidemic diarrhea virus
  Protein sequence of Spike (S) Protein for isolate USA/Colorado/2013 PEDV
MKSLTYFWLFLPVLSTLSLPQDVTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDTSVILAEGSIVLHTALGTNFSFVCSNSSNPHLATFAIPLGATQVPYYCFLKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYPISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALQLSARLESVEVNSMLTISEEALQLATISSFNGDGYNFTNVLGVSVYDPASGRVVQKRSFIEDLLFNKVVTNGLGTVDEDYKRCSNGRSVADLVCAQYYSGVMVLPGVVDAEKLHMYSASLIGGMVLGGFTSAAALPFSYAVQARLNYLALQTDVLQRNQQLLAESFNSAIGNITSAFESVKEAISQTSKGLNTVAHALTKVQEVVNSQGAALTQLTVQLQHNFQAISSSIDDIYSRLDILSADVQVDRLITGRLSALNAFVAQTLTKYTEVQASRKLAQQKVNECVKSQSQRYGFCGGDGEHIFSLVQAAPQGLLFLHTVLVPSDFVDVIAIAGLCVNDEIALTLREPGLVLFTHELQNHTATEYFVSSRRMFEPRKPTVSDFVQIESCVVTYVNLTRDQLPDVIPDYIDVNKTLDEILASLPNRTGPSLPLDVFNATYLNLTGEIADLEQRSESLRNTTEELQSLIYNINNTLVDLEWLNRVETYIKWPWWVWLIIFIVLIFVVSLLVFCCISTGCCGCCGCCCACFSGCCRGPRLQPYEVFEKVHVQ

SEQ ID NO: 24 - protein, 224 aa, Porcine epidemic diarrhea virus
  Protein sequence of ORF3 Protein for isolate USA/Colorado/2013 PEDV
MFLGLFQYTIDTVVKDVSKSANLSLDAVQELELNVVPIRQASNVTGFLFTSVFIYFFALFKASSLRRNYIMLAARFAVIVLYCPLLYYCGAFLDATIICCTLIGRLCLVCFYSWRYKNALFIIFNTTTLSFLNGKAAYYDGKSIVILEGGDHYITFGNSLVAFVSSIDLYLAIRGRQEADLQLLRTVELLDGKKLYVFSQHQIVGITNAAFDSIQLDEYATISE

SEQ ID NO: 25 - protein, 76 aa, Porcine epidemic diarrhea virus
  Protein sequence of the Envelope Protein (E) for isolate USA/Colorado/2013 PEDV
MLQLVNDNGLVVNVILWLFVLFFLLIISITFVQLVNLCFTCHRLCNSAVYTPIGRLYRVYKSYMQIDPLPSTVIDV

SEQ ID NO: 26 - protein, 226 aa, Porcine epidemic diarrhea virus
  Protein sequence of the Membrane Protein (M) for isolate USA/Colorado/2013 PEDV
MSNGSIPVDEVIQHLRNWNFTWNIILTILLVVLQYGHYKYSAFLYGVKMAILWILWPLVLALSLFDAWASFQVNWVFFAFSILMACITLMLWIMYFVNSIRLWRRTHSWWSFNPETDALLTTSVMGRQVCIPVLGAPTGVTLTLLSGTLLVEGYKVATGVQVSQLPNFVTVAKATTTIVYGRVGRSVNASSGTGWAFYVRSKHGDYSAVSNPSSVLTDSEKVLHLV

SEQ ID NO: 27 - protein, 441 aa, Porcine epidemic diarrhea virus
  Protein sequence of the Nucleocapsid Protein (N) for isolate USA/Colorado/2013 PEDV
MASVSFQDRGRKRVPLSLYAPLRVTNDKPLSKVLANNAVPTNKGNKDQQIGYWNEQIRWRMRRGERIEQPSNWHFYYLGTGPHADLRYRTRTEGVFWVAKEGAKTEPTNLGVRKASEKPIIPNFSQQLPSVVEIVEPNTPPTSRANSRSRSRGNGNNRSRSPSNNRGNNQSRGNSQNRGNNQGRGASQNRGGNNNNNNKSRNQSKNRNQSNDRGGVTSRDDLVAAVKDALKSLGIGENPDKLKQQQKPKQERSDSSGKNTPKKNKSRATSKERDLKDIPEWRRIPKGENSVAACFGPRGGFKNFGDAEFVEKGVDASGYAQIASLAPNVAALLFGGNVAVRELADSYEITYNYKMTVPKSDPNVELLVSQVDAFKTGNAKPQRKKEKKNKRETTQQLNEEAIYDDVGVPSDVTHANLEWDTAVDGGDTAVEIINEIFDTGN

SEQ ID NO: 28 - protein, 20 aa, Porcine epidemic diarrhea virus
PKGENSVAACFGPRGGFKNF

SEQ ID NO: 29 - protein, 24 aa, Porcine epidemic diarrhea virus
GKNTPKKNKSRATSKERDLKDIPE

SEQ ID NO: 30 - protein, 20 aa, Porcine epidemic diarrhea virus
TTQQLNEEAIYDDVGVPSDV